(19) United States
(12) Patent Application Publication
(10) Pub. No.: US 2025/0161477 A1
(43) Pub. Date: May 22, 2025

(54) NOVEL METHODS OF THERAPY

(71) Applicant: BIVICTRIX LIMITED, Macclesfield (GB)

(72) Inventor: Tiffany Thorn, Macclesfield (GB)

(21) Appl. No.: 18/836,995

(22) PCT Filed: Feb. 17, 2023
(86) PCT No.: PCT/GB2023/050367

§ 371 Date: Aug. 8, 2024

(30) Foreign Application Priority Data
Feb. 17, 2022 (GB) 2202170.3

(51) Int. Cl. A61K 47/68; A61P 35/00; C07K 16/28

(52) U.S. Cl. CPC A61K 47/6879; A61K 47/68031; A61P 35/00; C07K 16/2803; C07K 2317/31; C07K 2317/55; C07K 2317/622; C07K 2317/73

(57) ABSTRACT

The present invention relates to compositions for use in the non-immune suppressing treatment of a malignancy, wherein the composition comprises an agent that binds CD33 and CD56. The present invention also relates to combinations of agents targeting the protein pairs.

### TECHNICAL FIELD OF INVENTION

The present invention relates to a composition for use in the treatment of a malignancy wherein the composition comprises an agent that binds to CD33 and CD56. The present invention also relates to combinations of agents that bind to CD33 and CD56 and methods for identifying compositions for use in the treatment of malignancies.

### BACKGROUND TO THE INVENTION

Haematologic malignancies are forms of cancer that begin in the cells of blood-forming tissue, such as the bone marrow, or in the cells of the immune system. Examples of haematologic malignancies are acute and chronic leukaemias, lymphomas, multiple myeloma and myelodysplastic syndromes. While uncommon in solid tumours, chromosomal translocations are a common cause of these diseases. This commonly leads to a different approach in diagnosis and treatment of haematological malignancies. Unfortunately, the majority of patients who suffer from a haematologic malignancy live with an incurable disease.

One of the many potential side effects of haematological malignancies and their treatments is a suppressed immune system, or immunosuppression. A specific type of immunosuppression, myelosuppression is a condition in which bone marrow activity is decreased, resulting in fewer red blood cells, white blood cells, and platelets. Myelosuppression is often a side effect of some cancer (or other) treatments. Myelosuppression is problematic and potentially very dangerous for patients. Myelosuppression is one of the most common safety concerns in single antigen directed therapies. For example, Gemtuzumab ozogamicin is an immunoconjugate of an anti-CD33 antibody and a toxic calicheamicin-yl derivative. One of the major side-effects of Gemtuzumab ozogamicin includes myelosuppression.

It is an object of the present invention to provide therapies for diseases in which specific cell surface protein pairs, i.e. CD33 and CD56, are implicated. It is also an object of the invention to provide therapies for diseases in which the specific cell surface protein pairs are implicated which do not result in immunosuppression and/or myelosuppression. It is a further object of the invention to provide methods of determining which specific therapies targeting specific cell surface protein pairs would be successful in preventing immunosuppression and/or myelosuppression and/or impaired immune function. It would be desirable to identify a therapy which can be used for the treatment of a malignancy (such as a haematological cancer, Multiple Myeloma or Acute Myeloid Leukaemia (AML)) which is non-immune suppressing.

### SUMMARY OF THE INVENTION

The inventor has surprisingly shown that targeting both CD33 and CD56 on tumour cells provides an effective way of treating cancer as the dual targeting of this specific antigen pair targets tumour associated cells, but not healthy cells.

In one aspect, the invention relates to a composition for use in the treatment of a malignancy, e.g. cancer, wherein the composition comprises an agent that binds to CD33 and CD56.

In another aspect, the invention relates to a bispecific antibody or antibody fragment capable of binding CD33 and CD56 for use in the treatment of a malignancy, e.g. cancer.

In another aspect, the invention relates to a method for treating a malignancy, e.g. cancer, comprising administering to a subject in deed thereof an agent that binds to CD33 and CD56.

In another aspect, the invention relates to a method of targeting cells that express both CD33 and CD56 comprising administering to a subject an agent that binds to CD33 and CD56.

In another aspect, the invention relates to a combination of agents for use in the non-immune suppressing treatment of a malignancy, e.g. cancer, wherein the agents bind to CD33 and CD56 In another aspect, the invention relates to a bispecific antibody or antigen binding fragment thereof capable of binding CD33 and CD56.

In another aspect, the invention relates to a pharmaceutical composition comprising an antibody or antigen binding fragment thereof as described herein.

In another aspect, the invention relates to a kit comprising an antibody as described herein.

The following relates to all aspects above:

The treatment may be a non-immune suppressing treatment.

The non-immune suppressing treatment may be non-myelosuppressing treatment.

The agent may be an antibody or antigen binding fragment thereof.

The agent may be a bispecific antibody or antigen binding fragment thereof that binds CD33 and CD56 The composition may further comprises a payload.

The payload may be a cell killing agent, an immune-modulating payload, a macrophage class switching agent, or a light activatable payload.

The immune-modulating payload may be a STING agonist or a toll-like receptor agonist.

The cell killing agent may comprise a cytotoxin.

The cytotoxin may be selected from:
i) a peptide toxin; or
ii) a chemical toxin.

The composition may further comprise a linker for linking the payload to the agent that binds to CD33 and CD56 expressed on the cell surface.

The composition may be a bispecific antibody drug conjugate.

The malignancy may be a haematological cancers or Multiple Myeloma.

The malignancy may be AML or AML derived cancer.

### DETAILED DESCRIPTION

The embodiments of the invention will now be further described. In the following passages, different embodiments are described. Each aspect so defined may be combined with any other aspect or aspects unless clearly indicated to the contrary.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012); Therapeutic Monoclonal Antibodies: From Bench to Clinic, Zhiqiang An (Editor), Wiley, (2009); and Antibody Engineering, 2nd Ed., Vols 1 and 2, Ontermann and Dubel, eds., Springer-Verlag, Heidelberg (2010).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients. Suitable assays to measure the properties of the molecules disclosed herein are also described in the examples.

In accordance with a first aspect of the present invention, there is provided a composition for use in the treatment of a malignancy, wherein the composition comprises an agent, for example a cell inhibiting agent, that binds to CD33 and CD56.

The treatment is a non-immune suppressing treatment. Preferably, the non-immune supressing treatment of the malignancy is a non-myelosuppressing treatment.

The inventor has evaluated co-expression of antigens on AML cells and healthy cells. The inventor has surprisingly found that cancer cells express both CD33 and CD56 antigens on their cell surface whilst healthy cells do not express both antigens. This makes it possible to selectively target tumour cells.

Thus, unexpectedly, and advantageously, the present inventor has identified that cancer cells presenting both CD33 and CD56 antigens on their cell surface can be targeted with one or more agents which can bind to both proteins without any or reduced immune suppression. In contrast, the inventor has shown that agents targeting malignant cells expressing CD25 and CD34; or CD56 and CD7; or CD56 and CD11c; or CD33 and CD371 are not suitable as treatments for targeting malignant cells expressing those antigens as such targeting would result in immune suppression and/or myelosuppression and/or impaired immune function. This is because these target pairs were not only expressed on malignant cells, but also on healthy cells.

These results presented herein thus show that targeting CD33 and CD56 with a bispecific antibody or antigen binding fragment thereof targets cancerous cells expressing both CD33 and CD56 avoids targeting healthy haematological cell populations. Therefore, targeting both CD33 and CD56 avoids or reduces off-target cytotoxic effects. Off-target cytotoxicity that targets healthy cells, e.g. PBMC and/or BMMC, leads to immune suppression and/or myelosuppression and/or impaired immune function which are common side effects of anti-cancer chemotherapy. Targeting both CD33 and CD56 antigens which are expressed on the surface of cancerous cells but are not expressed on healthy haematological cells therefore avoids or reduces such off-target cytotoxicity of these healthy cells. This reduces immune suppression and/or myelosuppression and/or impairment of immune function.

Targeting the cells and treating cancer can be achieved with agents as described herein, for example bispecific antibodies.

The composition of the invention may be or comprise an antibody or antigen binding fragment thereof. Thus, in one embodiment the agent is an antibody or antigen binding fragment thereof. In one embodiment, the agent is a bispecific antibody or antigen binding fragment thereof capable of binding to both CD33 and CD56.

The composition may further comprise a payload for example a cell killing agent, an immune-modulating payload, a macrophage class switching agent or a light activatable payload. In certain embodiments, the cell killing agent comprises a cytotoxin. The cytotoxin may be selected from: i) a peptide toxin; or ii) a chemical toxin. The cytotoxin may be selected from: i) a peptide toxin ii) a chemical toxin, iii) an inhibitor of Bcl-2 or Bcl-axl, iv) an RNA Polymerase inhibitor such as α-amanitin, v) a spliceosome inhibitor, vi) a microtubule-targeting payload, or vii) a DNA-damaging payload. The skilled addressee will appreciate that a range of toxins will be compatible with the composition. Preferably the cell killing agent is auristatin MMAF. Suitable toxins are further exemplified herein.

The composition may further comprise a linker for linking the payload, e.g. the cell killing agent to the agent, e.g. cell inhibiting agent and/or antibody or antibody fragment, that binds to the pair of proteins expressed on the cell surface. Preferably the linker is a non-cleavable maleimidoca-proyl (mc) linker. Suitable linkers are further exemplified herein.

Thus, the agent may be or comprise an antibody drug conjugate (ADC).

In certain embodiments, the composition comprises a multispecific, e.g. bispecific, antibody drug conjugate. In an embodiment, the composition comprises or consists of a bispecific antibody or antibody fragment drug conjugate. In another embodiment, the composition is a trispecific antibody drug conjugate. In such an embodiment, the antibody binds CD33, CD56 and a further antigen target. For example, a half life extending moiety may be included which binds human serum albumin.

The skilled addressee will appreciate that the composition could be used for treating a number of diseases in which CD33 and CD56 are implicated. The disease in which CD33 and CD56 are implicated may be a malignant cancer. The malignant cancer may be selected from one of the following: haematological cancers or Multiple Myeloma or AML.

In accordance with a second aspect of the present invention, there is provided a combination of agents, for example cell inhibiting agents, for use in the non-immune suppressing treatment of a malignancy, wherein the cell inhibiting agents binds CD33 and CD56.

Preferably, the combination is for use in the non-myelosuppressing treatment of a malignancy.

The agents may comprise antibodies or antigen binding fragment thereof. In certain embodiments, the cell inhibiting agent may further comprise a payload, for example a cell killing agent, an immune-modulating payload or a light activatable payload. The cell killing agent may comprise a cytotoxin. The cytotoxin may be selected from: i) a peptide toxin; or ii) a chemical toxin. The cytotoxin may be selected from: i) a peptide toxin ii) a chemical toxin, iii) an inhibitor of Bcl-2 or Bcl-axl, iv) an RNA Polymerase inhibitor such as α-amanitin, v) a spliceosome inhibitor, vi) a microtubule-targeting payload, or vii) a DNA-damaging payload. The skilled addressee will appreciate that a range of toxins will be compatible with the agents. Preferably the cell killing agent is auristatin MMAF. Suitable toxins are further exemplified herein.

The agents may further comprise a linker for linking the payload, e.g. cell killing agent, to the cell inhibiting agent that binds to at least one of the CD33 or CD56 proteins expressed on the cell surface. Preferably the linker is a non-cleavable maleimidoca-proyl (mc) linker. Suitable linkers are further exemplified herein.

Thus, the agent may be or comprise an antibody drug conjugate (ADC).

The disease in which CD33 and CD56 are implicated may be a malignant cancer. The cancer may be selected from one of the following: haematological cancers or Multiple Myeloma or AML.

In accordance with a third aspect of the present invention, there is provided an agent, for example a cell inhibiting agent, for use in the non-immune suppressing treatment of a malignancy, wherein the cell inhibiting agent bispecifically binds to CD33 and CD56.

The agent may be for use in the non-myelosuppressing treatment of a malignancy.

The agent may be an antibody or antigen binding fragment thereof. The cell inhibiting agent further comprises a payload, for example a cell killing agent, an immune-modulating payload or a light activatable payload.

The agent may comprise antibodies or antigen binding fragment thereof. In certain embodiments, the cell inhibiting agent may further comprise a payload, e.g. a cell killing agent.

The cell killing agent may comprise a cytotoxin. The cytotoxin may be selected from: i) a peptide toxin; or ii) a chemical toxin. The cytotoxin may be selected from: i) a peptide toxin ii) a chemical toxin, iii) an inhibitor of Bcl-2 or Bcl-axl, iv) an RNA Polymerase inhibitor such as α-amanitin, v) a spliceosome inhibitor, vi) a microtubule-targeting payload, or vii) a DNA-damaging payload. The skilled addressee will appreciate that a range of toxins will be compatible with the agents. Preferably the cell killing agent is auristatin MMAF. Suitable toxins are further exemplified herein.

The agent may further comprise a linker for linking the payload, e.g. cell killing agent to the agent that binds to at least one of the pair of proteins expressed on the cell surface.

The cell inhibiting agent may be an antibody drug conjugate. Preferably the linker is a non-cleavable maleimidoca-proyl (mc) linker. Suitable linkers are further exemplified herein.

The agent may thus be or comprise an antibody drug conjugate (ADC).

The disease in which CD33 and CD56 are implicated may be a malignant cancer. The cancer may be selected from one of the following: haematological cancers or Multiple Myeloma or AML.

In another aspect, the invention provides a bispecific antibody or fragment thereof that is capable of binding CD33 and CD56 for use in the treatment of cancer. Also provided is a method of treating cancer comprising administering to a subject in need thereof a bispecific antibody fragment thereof capable of binding CD33 and CD56. As explained herein, the antibody bispecific antibody fragment thereof may be linked to payload, for example a cell killing agent, an immune-modulating payload or a light activatable payload.

In another aspect, the invention provides a bispecific antibody or fragment thereof capable of binding CD33 and CD56. As explained herein, bispecific antibody or fragment thereof may linked, e.g. conjugated to a payload, for example a cell killing agent, macrophage class switching agent, an immune-modulating payload or a light activatable payload.

The invention also provides a nucleic acid encoding a bispecific antibody or fragment thereof as described herein.

The invention also provides a host cell expressing a nucleic acid encoding a bispecific antibody or fragment thereof as described herein. The host cell may be a bacterial, viral, insect, plant, mammalian or other suitable host cell. In one embodiment, the cell is an E. coli cell. In another embodiment, the cell is a yeast cell. In another embodiment, the cell is a Chinese Hamster Ovary (CHO) cell.

The term bispecific refers to an antibody which binds to two different antigens, i.e. CD33 and CD56.

In another aspect, the invention provides a pharmaceutical composition comprising a bispecific antibody fragment thereof capable of binding CD33 and CD56.

In another aspect, the invention provides a kit comprising bispecific antibody fragment thereof capable of binding CD33 and CD56 and optionally instructions for use of said kit.

In another aspect, the invention provides an in vivo, in vitro or ex vivo method of targeting cells that express both CD33 and CD56 comprising administering to a subject an agent, e.g. a bispecific antibody or fragment thereof, that binds to CD33 and CD56.

In another aspect, the invention provides a method for reducing off target toxicity of a cancer treatment comprising administering to a subject an agent, e.g. a bispecific antibody fragment thereof, that binds to CD33 and CD56.

The antibody or antigen binding fragments thereof, or cell inhibiting agents, as herein above described with reference to all aspects may be for use in the treatment of a CD33+CD56+ malignancy, such as a haematological malignancy and/or a cancer.

The antibody or antigen binding fragments thereof, or agents, e.g. cell inhibiting agents, as herein above described with reference to all aspects may be for a method of treating a malignancy in an individual in need therefore, where the method comprises administering the antibody or antigen binding fragments thereof. Thus, the invention also relates to a method of treating cancer comprising administering to an individual in need thereof an agent, e.g. a bispecific antibody fragment thereof, that binds to CD33 and CD56.

According to the various aspects of the invention, it is preferred that the antibody or antigen binding fragments, or agents, e.g. cell inhibiting agents, thereof are artificially generated.

According to the various aspects of the invention, the antibody or antigen binding fragments thereof, or agents, e.g. cell inhibiting agents are isolated.

The term “isolated” refers to a moiety that is isolated from its natural environment. For example, the term “isolated” refers to an antibody, e.g. IgG1 that is substantially free of other antibodies or binding molecule, antibodies or antibody fragments. Moreover, an isolated antibody may be substantially free of other cellular material and/or chemicals.

In another related aspect of the present invention, there is provided antibody or antigen binding fragments thereof, or agents, e.g. cell inhibiting agents, as herein above described with reference to all aspects for use in the manufacture of a medicament for a malignancy.

As used herein “a medicament” refers to a substance used for medical treatment (i.e., a medicine). The medicament may be, e.g., a T cell product that is for use in adoptive cell transfer.

As used herein CD33 and CD56 is preferably human CD33 and CD56. In certain embodiments, the agents, bispecific antibodies or antigen binding fragments thereof specifically bind to CD33 and CD56 that are cell surface expressed. As used herein, the expression “cell surface-expressed” means CD33 and CD56 proteins that are expressed on the surface of a cell in vitro or in vivo, such that at least a portion of a CD33 and/or CD56 protein is exposed to the extracellular side of the cell membrane and is accessible to the bispecific antibody or antigen binding fragments thereof of the invention.

The term “malignancy” or “disease” refers to a malignancy characterised by the expression of both CD33 and CD56 proteins on the surface of the malignant cells (e.g., a malignancy that expresses CD33 and/or CD56 protein at levels considered acceptable for therapy with the antibody or antigen binding fragments thereof, or cell inhibiting agents, that specifically binds to CD33 and CD56).

As used herein for all embodiments of the invention, the term malignancy refers to a disease characterised by a cell surface or cell protein expression pattern as described above. Such a cell type may be any cell type of the human body and in particular a malignancy such as a cancer, including haematological cancers or Multiple Myeloma or AML.

It will be apparent to the skilled person that each protein may have a number of alternative names by which each protein is known. For example (non-exhaustive): CD33 may be known as: Siglec-3, sialic acid binding Ig-like lectin 3, SIGLEC3, SIGLEC-3, gp67, p67. CD56 may be known as: Neural cell adhesion molecule (NCAM), NCAM1, MSK39, NCAM, neural cell adhesion molecule 1.

CD33 is a 67 kDa plasma membrane protein that binds to sialic acid and is a member of the sialic acid-binding Ig-related lectin (SIGLEC) family of proteins. CD33 is known to be expressed on myeloid cells. CD33 expression has also been reported on a number of malignant cells.

Wild type human CD33 has been described, see e.g. UniProt Accession No. P20138).

An amino acid sequence for CD33 is shown below (SEQ ID No. 1).

MPLLLLLPLLWAGALAMDPNFWLQVQESVTVQEGLCVLVPCTFFH
PIPYYDKNSPVHGYWFREGAIISGDSPVATNKLDQEVQEETQGRF
RLLGDPSRNNCSLSIVDARRRDNGSYFFRMERGSTKYSYKSPQLS
VHVTDLTHRPKILIPGTLEPGHSKNLTCSVSWACEQGTPPIFSWL
SAAPTSLGPRTTHSSVLIITPRPQDHGTNLTCQVKFAGAGVTTER
TIQLNVTYVPQNPTTGIFPGDGSGKQETRAGVVHGAIGGAGVTAL
LALCLCLIFFIVKTHRRKAARTAVGRNDTHPTTGSASPKHQKKSK
LHGPTETSSCSGAAPTVEMDEELHYASLNFHGMNPSKDTSTEYSE
VRTQ

Thus, the agent, e.g. bispecific antibody or antibody fragment thereof, according to the various aspects of the invention binds to SEQ ID NO. 1 or a variant thereof. A variant has at least 80%, 85%, 90% or 95% sequence identity to SEQ D NO. 1.

CD33 plays a role in the regulation of cellular calcium influx necessary for the development, differentiation, and activation of B-lymphocytes.

Unless otherwise specified, the term CD33 as used herein refers to human CD33. CD33 is also known as “Membrane Spanning 4-Domains Al”, “Bp35” or “FMC7”, and these terms are used interchangeably and include variants and isoforms of human CD33.

The terms “CD33 binding molecule/protein/polypeptide/agent/moiety”, “CD33 antigen binding molecule molecule/protein/polypeptide/agent/moiety”, “anti-CD33 antibody”, “anti-CD33 antibody fragment”, “anti-CD33 antibody or antigen binding portion thereof” or “CD33 antibody” all refer to a molecule capable of specifically binding to the human CD33 antigen.

CD56, also known as Neural Cell Adhesion Molecule 1 (NCAM1) is over-expressed in many types of tumours, including neuroblastoma, multiple myeloma, small cell lung cancer, acute myeloid leukemia, Wilms tumour and ovarian cancer. Unless otherwise specified, the term CD33 as used herein refers to human CD56.

An amino acid sequence for CD56 is shown below (SEQ ID No. 2).

MLQTKDLIWTLFFLGTAVSLQVDIVPSQGEISVGESKFFLCQVAG
DAKDKDISWFSPNGEKLTPNQQRISVVWNDDSSSTLTIYNANIDD
AGIYKCVVTGEDGSESEATVNVKIFQKLMFKNAPTPQEFREGEDA
VIVCDVVSSLPPTIIWKHKGRDVILKKDVRFIVLSNNYLQIRGIK
KTDEGTYRCEGRILARGEINFKDIQVIVNVPPTIQARQNIVNATA
NLGQSVTLVCDAEGFPEPTMSWTKDGEQIEQEEDDEKYIFSDDSS
QLTIKKVDKNDEAEYICIAENKAGEQDATIHLKVFAKPKITYVEN
QTAMELEEQVTLTCEASGDPIPSITWRTSTRNISSEEKTLDGHMV
VRSHARVSSLTLKSIQYTDAGEYICTASNTIGQDSQSMYLEVQYA
PKLQGPVAVYTWEGNQVNITCEVFAYPSATISWFRDGQLLPSSNY
SNIKIYNTPSASYLEVTPDSENDFGNYNCTAVNRIGQESLEFILV
QADTPSSPSIDQVEPYSSTAQVQFDEPEATGGVPILKYKAEWRAV
GEEVWHSKWYDAKEASMEGIVTIVGLKPETTYAVRLAALNGKGLG
EISAASEFKTQPVHSPPPPASASSSTPVPLSPPDTTWPLPALATT
EPAKGEPSAPKLEGQMGEDGNSIKVNLIKQDDGGSPIRHYLVRYR
ALSSEWKPEIRLPSGSDHVMLKSLDWNAEYEVYVVAENQQGKSKA
AHFVFRTSAQPTAIPATLGGNSASYTFVSLLFSAVTLLLLC

Thus, the agent, e.g. bispecific antibody or antibody fragment thereof, according to the various aspects of the invention binds to SEQ ID NO. 2 or a variant thereof. A variant has at least 80%, 85%, 90% or 95% sequence identity to SEQ D NO. 2.

The terms “CD56 binding molecule/protein/polypeptide/agent/moiety”, “CD56 antigen binding molecule molecule/protein/polypeptide/agent/moiety”, “anti-CD56 antibody”, “anti-CD33 antibody fragment”, “anti-CD56 antibody or antigen binding portion thereof” or “CD56 antibody” all refer to a molecule capable of specifically binding to the human CD56 antigen.

The terms “antigen(s)” and “epitope(s)” are well established in the art and refer to the portion of a protein or polypeptide which is specifically recognized by a component of the immune system, e.g. an antibody or a T-cell/B-cell antigen receptor. As used herein, the term “antigen(s)” encompasses antigenic epitopes, e.g. fragments of antigens which are recognized by, and bind to, immune components. Epitopes can be recognized by antibodies in solution, e.g. free from other molecules. Epitopes can also be recognized by T-cell antigen receptors when the epitope is associated with a class I or class II major histocompatibility complex molecule.

The term “epitope” or “antigenic determinant” refers to a site on the surface of an antigen to which an immunoglobulin, antibody or antigen-binding fragment thereof specifically binds. Generally, an antigen has several or many different epitopes and reacts with many different antibodies. The term “specifically” includes linear epitopes and conformational epitopes.

Epitopes within protein antigens can be formed both from contiguous amino acids (usually a linear epitope) or non-contiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody or antigen-binding fragment thereof (i.e., epitope mapping) are well known in the art and include, for example, immunoblotting and immunoprecipitation assays, wherein overlapping or contiguous peptides are tested for reactivity with a given antibody or antigen-binding fragment thereof. Competition assays can also be used to determine if a test antibody binds to the same epitope as a reference antibody. Suitable competition assays are mentioned elsewhere herein and also shown in the examples. In some aspects, the epitope to which an antibody or antigen-binding fragment thereof binds can be determined by, e.g, NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g, site-directed mutagenesis mapping).

The term“binding molecule” “antibody” or “antigen binding molecule” as used herein refers to an immunoglobulin protein that is capable of binding an antigen target of interest, i.e. CD33 and CD56. In particular, the term “antibody” as used herein broadly refers to any polypeptide comprising complementarity determining regions (CDRs) that confer specific binding affinity of the polypeptide for an antigen. The term antibody as used herein encompasses polyclonal and monoclonal antibody preparations. The term “antibody” as used herein encompasses binding molecules with different antibody formats as well as antigen binding fragments.

The antibody or antigen-binding fragment thereof described herein, “which binds” or is “capable of binding” the antigen of interest, binds the antigen with sufficient affinity such that the antibody or antigen-binding fragment thereof is useful as a therapeutic or diagnostic agent in targeting CD33 and CD56 as described herein. The term “specific” may refer to the situation in which the antibody molecule will not show any significant binding to molecules other than its specific binding partner.

The terms “polypeptide(s)” and “protein(s)” are used interchangeably throughout the application and denote at least two covalently attached amino acids, thus may signify proteins, polypeptides, oligopeptides, peptides, and fragments thereof. The protein may be made up of naturally occurring amino acids and peptide bonds, or synthetic peptidomimetic structures. Hence, “amino acid(s)” or “peptide residue(s)”, as used herein, denote both naturally occurring and synthetic amino acids. In some cases, the immunoglobulin proteins of the present invention may be synthesized using any in vivo or in vitro protein synthesis technique known in the art.

The antibody or antigen binding fragments thereof, or agents, e.g. cell inhibiting agents, may be capable of inducing CD33 and CD56 receptor mediated internalisation into a CD33+ and/or CD56+ cell.

CD33+CD56+ malignancies include, but are not limited to, haematological cancers, AML and Multiple Myeloma.

The antibody or antigen binding fragments thereof, or agents, e.g. cell inhibiting agents, may bispecifically bind CD33 and CD56 and wherein the CD33+ and CD56+ cell is a malignant cell.

The antibody or antigen binding fragments thereof, or agents, e.g. cell inhibiting agents, may be capable of mediating antibody dependent cellular cytotoxicity.

The antibody or antigen binding fragment thereof, or agents, e.g. cell inhibiting agents, may be attached to, or formed with an immune effector cell. The immune effector cell may comprise a T cell and/or a NK cell. Preferably, immune effector cell is a T cell. The immune effector cell may be a bispecific anti-CD33 anti-CD56 CAR-T. Thus, the agent may be a CAR-T cell. The T cell may comprise a CD33+ T cell, a CD56+ T cell or a combination thereof.

The antibody or antigen binding fragment thereof, or agents, e.g. cell inhibiting agents may be a trispecific immune cell engager. The antibody or antigen binding fragment thereof, or cell inhibiting agents may additionally comprise an immune cell binding domain. The immune cell binding domain is able to attach to, or bind to, an immune cell. The immune cell binding domain may bind to one or more T cells or NK cells. The immune cell binding domain binds to or attaches to an immune cell causing the immune cell to kill the malignant cell to which the antibody, antigen binding fragment thereof or cell inhibiting agent is bound.

The composition, antibody drug conjugate, antibody or antigen binding fragments thereof, or agents, e.g. cell inhibiting agents, may comprise: i) a payload, for example a cell killing agent, an immune-modulating payload, a macrophage class switching agent or a light activatable payload; ii) a CD33 binding portion; and iii) a CD56 binding portion. The CD33 binding portion may be an antibody or antibody fragment thereof. The CD56 binding portion may be an antibody or antibody fragment thereof.

An antibody drug conjugate according to the various aspects of the invention shows preferential cytotoxicity for cells expressing both CD33 and CD56 over cells that express neither of these targets or cells that express only one of these targets.

An antibody drug conjugate according to the invention shows reduced off target cytotoxicity in a CD34+ to CD33+ myloid differentiation colony forming assay compared to a conjugated antibody that targets a single antigen. The assay may be performed as shown in the examples. An antibody drug conjugate according to the invention provides selective cell killing of dual positive CD33+CD56+ cells, e.g. in vivo or in a cytotoxicity assay as shown in the examples.

A bispecific antibody or fragment thereof as described herein is capable of binding both CD33 and CD56 and is also capable of mediating selective cytotoxicity.

In alternative embodiments, the CD33 and/or CD56 binding portion comprises an antigen binding fragment of an antibody, or individual cell inhibiting agents.

In embodiments of the various aspects of the invention, the agent, e.g. antibody or fragment thereof, e.g. bispecific antibody is linked/conjugated to a payload.

A skilled persons would know that different payloads for such conjugates are known in the art (see e.g. Gingrich J. How the Next Generation Antibody Drug Conjugates Expands Beyond Cytotoxic Payloads for Cancer Therapy—J. ADC. Apr. 7, 2020).

In one embodiment, the payload may be a cell killing agent.

In one embodiment, the payload may be an immune-modulating payload.

In one embodiment, the payload may be light activatable payload.

As used herein, an immune-modulating payload includes any moiety that modulates the immune system, for example which stimulates the immune system and/or kills the target cell. Thus, a moiety that has immuno-activating and/or antineoplastic activities can be used. Such moieties may be synthetic peptides that recognise the specific target and trigger (agonist) or block (antagonist) inflammatory responses. The target may be a pattern recognition receptor (PRR), including Toll-like receptors (TLRs), NOD-like receptors (NLRs), RIG-1-like receptors (RLRs), C-type lectin receptors (CLRs) and cytosolic dsDNA sensors (CDSs).

Examples of payloads include agonists for the stimulator of interferon genes protein (STING; transmembrane protein 173; TMEM173). Such payloads include cyclic dinucleotides and compounds listed in see WO2021113679). Activation of the STING pathway triggers an immune response that results in generation of specific killer T-cells that shrink tumours and can provide long-lasting immunity so the tumours do not recur. Alternatively, payloads that act on toll-like receptors (TLRs) may be used. For example, agonists that bind to TLR7 and/or TLR8 can be used.

Another example is a macrophage class switching agent.

A light activatable payload (IRDye® 700DX, IR700) may also be used. Light activation of the non-toxic payload results in the generation of singlet oxygen species that damage the cell membrane integrity, resulting in necrotic and immunogenic cell death of tumour cells, resulting in minimal damage to surrounding normal tissue.

The cell killing portion may be a cytotoxin and the skilled addressee will understand that a range of cytotoxins will be compatible with the composition. A cytotoxin, which may be selected from: i) a peptide toxin, or ii) a chemical toxin, or iii) an inhibitor of Bcl-2 or Bcl-axl, iv) an RNA Polymerase inhibitor such as α-amanitin, v) a spliceosome inhibitor, vi) a microtubule-targeting payload, or vii) a DNA-damaging payload. Preferably the cell killing agent is auristatin MMAF. The antibody or antigen binding fragments thereof, or cell inhibiting agents, may further comprise a linking portion linking the cell kill portion with the CD56 binding portion and/or the CD28 binding portion. The antibody or antigen binding fragments thereof, or cell inhibiting agents, may be in the format of an antibody drug conjugate.

In the embodiment of a bispecific antibody, then such an antibody may be a full-length antibody or antigen binding fragment.

As used herein, the terms “treat”, “treating” and “treatment” are taken to include an intervention performed with the intention of preventing the development or altering the pathology of a disorder or symptom. Accordingly, “treatment” refers to both therapeutic treatment and prophylactic or preventative measures, wherein the object is to prevent or slow down (lessen) the targeted disorder or symptom. Accordingly, the term “treating” encompasses treating and/or preventing the development of a disorder or symptom. As used herein, “therapy” refers to the prevention or treatment of a disease or disorder. Therapy may be prophylactic or therapeutic.

In such aspects, the antibody or antigen binding fragments thereof, or agents, e.g. cell inhibiting agents, of the invention are administered to a patient in remission from the malignancy, resulting in preventing or delaying recurrence of the underlying malignancy.

As used herein, a “patient”, “subject” or “individual” is typically a human who is undergoing treatment for, or has been diagnosed as having, malignancy, preferably a CD33+CD56+ malignancy, e.g. a cancer. In some embodiments, the antibody or antigen binding fragments thereof, or cell inhibiting agents, are administered to a patient in remission from a CD33+CD56+ malignancy, whereby the recurrence of the malignancy is prevented or delayed. In some embodiments, the patient lacks detectable cells of the malignancy. As used herein, a “lack of detectable cells” is determined by standard diagnostic or prognostic methods. A patient in remission from AML typically exhibits resolution of abnormal clinical features, return to normal blood counts and normal haematopoiesis in the bone marrow with <5% blast cells, a neutrophil count of >1.000-1,500, a platelet count of >100,000, and disappearance of the leukemic clone. See, e.g., The Merck Manual, Sec. 11, Ch. 138 (17th ed. 1997): Estey, 2001, Cancer 92(5): 1059-1073.

In some embodiments, the patient in remission from the CD33+CD56+ malignancy has not undergone a bone marrow transplant. In other embodiments, the patient in remission from the CD33+CD56+ malignancy has undergone a bone marrow transplant. The bone marrow transplant can be either an autologous or an allogeneic bone marrow transplant.

In embodiments treating a CD33+CD56+ malignancy and delaying preventing or delaying recurrence of CD33+CD56+ malignancy involves the inducing cancer cell death and/or inhibiting or reducing cancer cell growth.

As used in the various aspects and embodiments herein, the term “reduce” includes reduction by at least 10%, 20%, 30%, 40%, 50%, 60%, 70%, 80%, 90% or more.

The antibody or antigen binding fragments thereof, or agents, e.g. cell inhibiting agents, may be part of a composition (e.g., a therapeutic composition) that comprises the compound (i.e., the antibody or antigen binding fragments thereof, or cell inhibiting agents) and one or more other components. A composition may be a therapeutic/pharmaceutical composition that comprises the antibody or antigen binding fragments thereof, or agents, e.g. cell inhibiting agents, and a pharmaceutically acceptable excipient, adjuvant, diluent and/or carrier. Therapeutic compositions may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected compound without causing any undesirable biological effects or interacting in a deleterious manner with any of the other components of the pharmaceutical composition in which it is contained.

Excipients are natural or synthetic substances formulated alongside an active ingredient (e.g., the vaccine, cell cycle inhibitor, modulator of an immune suppression mechanism, or immune check point inhibitor (as appropriate)), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art.

Diluents are diluting agents. Pharmaceutically acceptable diluents are well known in the art. A suitable diluent is therefore easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

As used herein, the terms “effective amount” and “therapeutically effective amount” refer to the quantity of the active therapeutic agent sufficient to yield a desired therapeutic response without undue adverse side effects such as toxicity, irritation, or allergic response. The specific “effective amount” will, obviously, vary with such factors as the particular condition being treated, the physical condition of the patient, the type of animal being treated, the duration of the treatment, the nature of concurrent therapy (if any), and the specific formulations employed and the structure of the compounds or its derivatives. In this case, an amount would be deemed therapeutically effective if it resulted in one or more of, but not limited to, the following: (a) the inhibition of cancer cell growth; and (b) the killing of cancer cells.

The dose of the antibody or antigen binding fragments thereof, or agents, e.g. cell inhibiting agents, and therapeutic compositions thereof administered to a patient may vary depending upon the age and the size of the patient, target disease, conditions, route of administration, and the like. The preferred dose is typically calculated according to body weight or body surface area.

Methods of administration of the antibody or antigen binding fragments thereof, or cell inhibiting agents, and therapeutic compositions thereof include, but are not limited to, intradermal, intramuscular, intraperitoneal, intravenous, subcutaneous, intranasal, epidural, and oral routes. The antibody or antigen binding fragments thereof, or agents, e.g. cell inhibiting agents, and therapeutic compositions thereof may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The compositions and antibodies of the invention may be administered together with a second moiety for example a therapeutic molecule. Administration may be concurrent or sequential. The second moiety may be a chemotherapy agent, biologic, cytokine, small molecule, CAR-T therapy or radiotherapy treatment.

Chemotherapy agents include alkylating agents, plant alkaloids, antimetabolites, anthracyclines, topoisomerase inhibitors and corticosteroids. For example, the chemotherapy can include vinorelbine, cisplatin, carboplatin, gemcitabine, paclitaxel, topotecan, docetaxel, irinotecan, pemetrexed, etoposide, or any combination thereof.

A biologic may be an antibody therapy, for example an antibody that targets a checkpoint inhibitor, such as PD-1 (e.g. Pembrolizumab, Nivolumab or Cemiplimab), PD-L1 (e.g. Atezolizumab, Avelumab or Durvalumab), PD-L2, LAG-3 (e.g. Relatlimab), Tim-3 or CTLA4 (e.g. Ipilimumab).

The small molecule therapy may be Pexidartinib.

In another embodiment, the second moiety is a label, for example a fluorescent molecule, β-galactosidase, luciferase molecules, chemical dyes, fluorophores or a radioisotope.

In another aspect, the agents, antibodies or antigen-binding fragments thereof of the invention are modified to increase half-life, for example by a chemical modification, especially by PEGylation, or by incorporation in a liposome, or using a serum albumin protein or an antibody or antibody fragment that binds human serum albumin. Increased half-life can also be conferred by conjugating the molecule to an antibody fragment. The term “half-life” as used herein refers to the time taken for the serum concentration of the amino acid sequence, compound or polypeptide to be reduced by 50%, in vivo, for example due to degradation of the sequence or compound and/or clearance or sequestration of the sequence or compound by natural mechanisms.

Half-life may be increased by at least 1.5 times, preferably at least 2 times, such as at least 5 times, for example at least 10 times or more than 20 times, greater than the half-life of the corresponding antibodies of the invention. For example, increased half-life may be more than 1 hours, preferably more than 2 hours, more preferably more than 6 hours, such as more than 12 hours, or even more than 24, 48 or 72 hours, compared to the antibody of the invention. The in vivo half-life of an amino acid sequence, compound or polypeptide of the invention can be determined in any manner known per se, such as by pharmacokinetic analysis. Suitable techniques will be clear to the person skilled in the art. Half-life can for example be expressed using parameters such as the t1/2-alpha t1/2-beta and the area under the curve (AUC).

Preferably, the dual targeting therapy described herein will provide a benefit to the treatment of a CD33+CD56+ malignancy in a subject in need thereof. For example, the dual targeting therapy may have an additive or synergistic effect on the treatment of a malignancy in a subject in need thereof. A dual targeting therapy is defined as affording an “additive effect”, “synergistic effect” or a “synergistic treatment” if the effect is therapeutically superior, as measured by, for example, the extent of the response (e.g., apoptosis or cell viability), the response rate, the time to disease progression or the survival period, to that achievable on dosing one or other of the components of the dual targeting therapy at its conventional dose. For example, the effect of the dual targeting therapy is additive if the effect is therapeutically superior to the effect achievable with an antibody or antigen binding fragments thereof that specifically binds to CD33, or CD56 alone. For example, the effect of the combination treatment may be synergistic if the effect of the combination treatment supersedes the effect of the individual treatments added together. Further, the effect of the combination is beneficial (e.g., additive or synergistic) if a beneficial effect is obtained in a group of subjects that does not respond (or responds poorly) to a cell-inhibiting agent that specifically binds to CD33 alone or a cell-inhibiting agent that specifically binds to CD56 alone. In addition, the effect of the combination treatment is defined as affording a benefit (e.g. additive or synergistic effect) if one of the components is dosed at its conventional dose and the other component is dosed at a reduced dose and the therapeutic effect, as measured by, for example, the extent of the response, the response rate, the time to disease progression or the survival period, is equivalent to or better than that achievable on dosing conventional amounts of either one of the components of the combination treatment.

As used herein, “killing of a target cell” relates to an inhibition of protein synthesis, for example such that cell viability is reduced, or an induction of apoptosis resulting in elimination or death of target cells. Assays to determine cell killing and apoptosis are well known in the art. Cytotoxicity assays assess the number of live and dead cells in a population after treatment with a pharmacological substance (e.g., an LDH cytotoxicity assay, or a live-dead cell assay). Apoptosis assays assess how cells are dying by measuring markers that are activated upon cell death (e.g., a PS exposure assay, a caspase activation assay, a DNA fragmentation assay, a GSH/GSSG determination, a LDH cytotoxicity assay, a live-dead cell assay, or a non-caspase protease activation assay).

As used herein “inhibit the cell growth” (e.g., referring to target cells) refers to any measurable decrease in the growth or proliferation of a target cell when contacted with the antibody or antigen binding fragments thereof, or cell inhibiting agents, according to the present invention as compared to the growth of the same cell not in contact with the antibody or antigen binding fragments thereof, or cell inhibiting agents, according to the present disclosure, e.g., the inhibition of growth of a cell by at least about 10%, 20%, 30%, 40%, 50%, 60%, 70%, 80%, 90%, 99%, or 100%. Assays to determine cell viability or proliferation are well known in the art. Cell viability assays assess how healthy the cells are by measuring markers of cellular activity (e.g., an ATP and ADP determination assay, a cell cycle assay, a cell proliferation assay, a cell viability assay, an LHD cytotoxicity assay, or a live-dead cell assay). Cell proliferation assays assess the growth rate of a cell population or to detect daughter cells in a growing population (e.g., a cell cycle assay, a cell proliferation assay, a cell viability assay, or a senescence assay).

As used herein, “CD33 expressing cell” and “CD33+ cell” refers to a cell with CD33 as surface antigen. As used herein, “CD56 expressing cell” and “CD56+ cell” refers to a cell with CD56 as surface antigen. As used herein, “CD33 and CD56 expressing cell” and “CD33+CD56+ cell” refers to a cell with both CD33 and CD56 as surface antigens.

As used herein “target cell” refers to a cell or cell-type characterised by the expression or overexpression of the two target molecules CD33 and CD56. Any type of cell expressing both CD33 and CD56 may be envisaged as a target cell for treatment with the antibody or antigen binding fragments thereof, or cell inhibiting agents, of the invention. In certain embodiments, the cell is a tumour cell, for example a tumour cell from a malignancy.

In certain embodiments, the antibody or antigen binding fragments thereof, or agent, e.g. cell inhibiting agent, described herein are capable of inducing CD33, receptor mediated internalisation of said antibody or antigen binding fragments thereof into a CD33+ cell, and/or CD56 receptor mediated internalisation of said the antibody or antigen binding fragments thereof, or agents, e.g cell inhibiting agents, into a CD56+ cell. In certain embodiments, the antibody or antigen binding fragments thereof, or agent, e.g. cell inhibiting agent, is an antibody or antigen binding fragments thereof that specifically binds to both CD33 and CD56 and is capable of inducing internalisation of the agent into a CD33+CD56+ cell upon binding of both CD33 and CD56 on a cell surface.

As used herein, “CD33 receptor mediated internalisation” refers to being taken up by (i.e., entry of) a CD33+ cell upon binding to CD33 on the cell surface. For therapeutic applications, internalisation in vivo is contemplated. As used herein, “CD56 receptor mediated internalisation” refers to being taken up by (i.e., entry of) a CD56+ cell upon binding to CD56 on the cell surface. For therapeutic applications, internalisation in vivo is contemplated.

For therapeutic applications, the concentration of the antibodies or antigen binding fragments or agents, e.g. cell inhibiting agents, employed should be sufficient for the antibody or antigen binding fragments or cell inhibiting agents to be internalised and kill an CD33+CD56+ cancer cell. Depending on the potency of the antibody or antigen binding fragments thereof, or cell inhibiting agents, in some instances, the uptake of a single molecule into the cell is sufficient to kill the target cell to which the agent binds.

In certain embodiments, the antibody or antigen binding fragments thereof, or cell inhibiting agents, of the invention may be antibody drug conjugates (ADCs), small-molecule drug conjugates (SMDCs), immunotoxins, peptide and non-peptide conjugates, imaging agents, therapeutic vaccines, nanoparticles.

The terms “antibody” or “antibodies” as used herein refer to molecules or active fragments of molecules that bind to known antigens, particularly to immunoglobulin molecules and to immunologically active portions of immunoglobulin molecules, i.e., molecules that contain a binding site that immunospecifically binds an antigen (i.e., CD33, or CD56). The immunoglobulin according to the invention can be of any class (IgG, IgM, IgD, IgE, IgA and IgY) or subclass (e.g., IgG1, IgG2, IgG3, IgG4, IgA1 and IgA2) or subclasses (isotypes) of immunoglobulin molecule (e.g., IgG in IgG1, IgG2, IgG3, and IgG4, or IgA in IgA1 and IgA2).

As antibodies can be modified in a number of ways, the term “antigen-binding protein” or “antibody” should be construed as covering antibody fragments, derivatives, functional equivalents and homologues of antibodies, including any polypeptide comprising an immunoglobulin binding domain such as described herein. The terms also extends to different antibody formats, such as formats containing a Fab region and an scFV region as in specific aspects of the invention.

In a full-length antibody, each heavy chain is comprised of a heavy chain variable region or domain (abbreviated herein as HCVR, VH or V_{H}) and a heavy chain constant region. The heavy chain constant region is comprised of three domains, C_{H}1, C_{H}2 and C_{H}3. Each light chain is comprised of a light chain variable region or domain (abbreviated herein as LCVR, VL or V_{L}) and a light chain constant region. The light chain constant region is comprised of one domain, CL.

Antibodies may include the kappa (κ) and lambda (A) light chains and the alpha (IgA), gamma (IgG1, IgG2, IgG3, IgG4), delta (IgD), epsilon (IgE) and mu (IgM) heavy chains, or their equivalents in other species. Full-length immunoglobulin “light chains” (usually of about 25 kDa or usually about 214 amino acids long) consist of a variable region of approximately 110 amino acids at the NH2-terminus and a kappa or lambda constant region at the COOH-terminus. Full-length immunoglobulin “heavy chains” (usually of about 50 kDa or 446 amino acids long), likewise consist of a variable region (of about 116 amino acids) and one of the aforementioned heavy chain constant regions, e.g. gamma (of about 330 amino acids).

Light or heavy chain variable regions are generally composed of a “framework” region (FR) interrupted by three hypervariable regions, also called CDRs. The extent of the framework region and CDRs have been precisely defined. The sequences of the framework regions of different light and heavy chains are relatively conserved within a species. The framework region of an antibody, i.e. the combined framework regions of the constituent light and heavy chains, serves to position and align the CDRs. The CDRs are primarily responsible for binding to an epitope of an antigen. There are three CDRs in each of the variable regions of the heavy chain and the light chain, which are designated CDR1, CDR2 and CDR3, for each of the variable regions. The term “CDR set” refers to a group of three CDRs that occur in a single variable region capable of binding the antigen. The exact boundaries of these CDRs can be defined differently according to different systems known in the art.

Different definitions of the CDRs are commonly in use. The method described by Kabat is the most commonly used and CDRs are based on sequence variability (Kabat et al., (1971) Ann. NY Acad. Sci. 190:382-391 and Kabat, et al., (1991) Sequences of Proteins of Immunological Interest, Fifth Edition, U.S. Department of Health and Human Services, NIH Publication No. 91-3242). Chothia refers instead to the location of the structural loops (Chothia and Lesk, J. Mol. Biol. 196:901-917 (1987)). The Kabat numbering system is generally used when referring to a residue in the variable domain (approximately residues 1-107 of the light chain and residues 1-113 of the heavy chain). Another system is the ImMunoGeneTics (IMGT) numbering scheme (Lefranc et al., Dev. Comp. Immunol., 29, 185-203 (2005)). According to the IMGT numbering scheme, a CDR is a loop region of a variable domain, delimited according to the IMGT unique numbering for V domain. There are three CDR-IMGT in a variable domain: CDR1-IMGT (loop BC), CDR2-IMGT (loop C′C″), and CDR3-IMGT (loop FG).

Heavy chain CDRs are designated HCDR1, HCDR2 and HCDR3. Light chain CDRs are designated LCDR1, LCDR2 and LCDR3.

“Framework” or “FR” refers to variable domain residues other than hypervariable region (UVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

In one embodiment, the antibody is comprised of four polypeptide chains, two heavy (H) chains and two light (L) chains, or any functional fragment, mutant, variant, or derivation thereof, which retains the essential epitope binding features of an Ig molecule.

The term “antibody” is not only inclusive of antibodies generated by methods comprising immunisation, but also includes any polypeptide, e.g., a recombinantly expressed polypeptide, which is made to encompass at least one CDR capable of specifically binding to an epitope on an antigen of interest. Hence, the term applies to such molecules regardless whether they are produced in vitro, in cell culture, or in vivo. Methods of producing polyclonal and monoclonal antibodies are known in the art.

It is possible to take monoclonal and other antibodies and use techniques of recombinant DNA technology to produce other antibodies or chimeric molecules which generally retain the specificity of the original antibody. Such techniques may involve introducing the CDRs into a different immunoglobulin framework, or grafting variable regions onto a different immunoglobulin constant regions. Alternatively, a hybridoma or other cell producing an antibody molecule may be subject to genetic mutation or other changes, which may or may not alter the binding specificity of antibodies produced.

Within the scope of the present invention the terms “antibody” or “antibodies” include human and humanised antibodies as well as active fragments thereof. Examples of active fragments of molecules that bind to known antigens include Fab, Fab′, F(ab′)2, F(ab′)3, Fabc, Fd, single chain Fv (scFv), (scFv)2, Fv, scFv-Fc, heavy chain only antibody, diabody, tetrabody, triabody, minibody, or antibody mimetic protein,including the products of a Fab immunoglobulin expression library and epitope-binding fragments of any of the antibodies and fragments mentioned above. Thus, the antibody fragment/antigen-binding fragment may comprise or consist of any of these fragments.

The “Fab fragment” of an antibody (also referred to as fragment antigen binding) contains the constant domain (CL) of the light chain and the first constant domain (CHI) of the heavy chain along with the variable domains VL and VH on the light and heavy chains respectively. The variable domains comprise the complementarity determining loops (CDR, also referred to as hypervariable region) that are involved in antigen binding. Fab′ fragments differ from Fab fragments by the addition of a few residues at the carboxy terminus of the heavy chain CHI domain including one or more cysteines from the antibody hinge region.

As used herein, the term “single-chain” refers to a molecule comprising amino acid monomers linearly linked by peptide bonds. In certain embodiments, one of the antigen binding moieties, e.g., antigen binding polypeptide construct, is a single-chain Fab molecule, i.e. a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule. Fv fragments (˜25 kDa) consist of the two variable domains, VH and VL. Naturally, VH and VL domain are non-covalently associated via hydrophobic interaction and tend to dissociate. However, stable fragments can be engineered by linking the domains with a hydrophilic flexible linker to create a single chain Fv (scFv). In certain other embodiments, one of the antigen binding moieties is a single-chain Fv molecule (scFv).

“Single-chain Fv” or “scFv” antibody fragments comprise the VH and VL domains of an antibody, wherein these domains are present in a single polypeptide chain. In one embodiment, the Fv polypeptide further comprises a polypeptide linker between the VH and VL domains which enables the scFv to form the desired structure for antigen binding.

The term “Fc” or “Fc domain” or “Fc region” or “Fc construct” herein is used to define a C-terminal region of an immunoglobulin heavy chain. The term includes native sequence Fc regions and variant Fc regions.

“Fc region”, as used herein, generally refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences.

The Fc sequence of an immunoglobulin generally comprises two constant domains, a CH2 domain and a CH3 domain, and optionally comprises a CH4 domain. By “Fc polypeptide” herein is meant one of the polypeptides that make up an Fc region. An Fc polypeptide may be obtained from any suitable immunoglobulin, such as IgG1, IgG2, IgG3, or IgG4 subtypes, IgA, IgE, IgD or IgM. In some embodiments, an Fc polypeptide comprises part or all of a wild type hinge sequence (generally at its N terminus). In some embodiments, an Fc polypeptide does not comprise a functional or wild type hinge sequence. The antibody may comprise a CH2 domain. The CH2 domain is for example located at the N-terminus of the CH3 domain, as in the case in a human IgG molecule. The CH2 domain of the antibody is in one embodiment the CH2 domain of human IgG1, IgG2, IgG3, or IgG4, e.g the CH2 domain of human IgG1. The sequences of human IgG domains are known in the art.

As used herein the term “humanised antibody” or “humanised version of an antibody” refers to antibodies in which the framework or “complementarity determining regions” (CDR) have been modified to comprise the CDR of an immunoglobulin of different specificity as compared to that of the parent immunoglobulin. In some exemplary embodiments, the CDRs of the VH and VL are grafted into the framework region of human antibody to prepare the “humanised antibody.” See e.g., Riechmann, L., et al., Nature 332 (1988) 323-327; and Neuberger, M. S., et al., Nature 314 (1985) 268-270. The heavy and light chain variable framework regions can be derived from the same or different human antibody sequences. The human antibody sequences can be the sequences of naturally occurring human antibodies. Human heavy and light chain variable framework regions are listed e.g., in Lefranc, M.-P., Current Protocols in Immunology (2000)-Appendix 1P A.1P.1-A.1P.37 and are accessible via IMGT, the international ImMunoGeneTics information System® (http://imgt.cines.fr) or via http://vbase.mrc-cpe.cam.ac.uk, for example. Optionally the framework region can be modified by further mutations. Exemplary CDRs correspond to those representing sequences recognising the antigens noted above for chimeric antibodies. In some embodiments, such humanised version is chimerised with a human constant region. The term “humanised antibody” as used herein also comprises such antibodies which are modified in the constant region to generate the properties according to the disclosure, especially in regard to C1q binding and/or FcR binding, e.g., by “class switching” i.e., change or mutation of Fc parts (e.g., from IgG1 to IgG4 and/or IgG1/IgG4 mutation).

As used herein the term “human antibody” is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunisation, of producing a full repertoire or a selection of human antibodies in the absence of endogenous immunoglobulin production. Transfer of the human germ-line immunoglobulin gene array in such germ-line mutant mice results in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Brueggemann, M. D., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole, A., et al. and Boerner, P., et al. are also available for the preparation of human monoclonal antibodies (Cole, A., et al., Monoclonal Antibodies and Cancer Therapy, Liss, A. R. (1985) p. 77; and Boerner, P., et al., J. Immunol. 147 (1991) 86-95). As already mentioned, according to the instant disclosure the term “human antibody” as used herein also comprises such antibodies which are modified in the constant region to generate the properties according to the disclosure, for example in regard to C1q binding and/or FcR binding, e.g., by “class switching” i.e., change or mutation of Fc parts (e.g., from IgG1 to IgG4 and/or IgG1/IgG4 mutation).

As used herein the term “antibody fragment” refers to a portion of a full-length antibody, the term “antigen binding fragments” refers to a variable domain thereof, or at least an antigen binding site thereof, for example the CDRs. Examples of antibody fragments include diabodies, single-chain antibody molecules, and multispecific antibodies formed from antibody fragments. scFv antibodies are, e.g., described in Huston, J. S., Methods in Enzymol. 203 (1991) 46-88. Antibody fragments can be derived from an antibody of the present invention by a number of art-known techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

As used herein the term “bispecific antibodies” refers to antibodies that bind to two (or more) different antigens. A bispecific antibody typically comprises at least two different variable domains, wherein each variable domain is capable of specifically binding to a separate antigen. In certain aspects, the bispecific antibodies of the invention are human antibodies. As used herein, the expression “bispecific antibody” means a protein, polypeptide or molecular complex comprising at least a first antigen-binding domain and a second antigen-binding domain. Each antigen-binding domain within the bispecific antibody comprises at least one CDR that alone, or in combination with one or more additional CDRs, specifically binds to a particular antigen. In the context of the present invention, the first antigen-binding domain specifically binds a first antigen (e.g., CD33), and the second antigen-binding domain specifically binds a second, distinct antigen (e.g., CD56). In certain aspects, the bispecific molecules are capable of simultaneously binding to human CD33 and human CD56.

In certain embodiments the bispecific antibodies may be referred to as “anti-CD33×CD56” or “anti-CD33/anti-CD56” and so forth. A bispecific antibody may have a sequence as shown in the examples. The CD33 binding portion may comprise Gemtuzumab or a fragment thereof. The CD33 binding portion may comprise SEQ ID. 3 and/or 4 or SEQ ID. 7 and/or 8.

A bispecific antibody may have a drug-to-antibody ratio (DAR) of 3 to 7, e.g. 3, 4, 5, 6, or 7.

Any bispecific antibody format or technology may be used to make the bispecific antibodies of the present invention. Specific exemplary bispecific formats that can be used in the context of the present invention include, without limitation, e.g., scFv-based or diabody bispecific formats, IgG-scFv fusions, Fc-Fab-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, Mab2 bispecific formats (see, e.g., Klein et al. 2012, imAbs 4:6, 1-11, and references cited therein, for a review of the foregoing formats) and Fab-based bispecific formats. In certain embodiments, the bispecific antibody is a Fab-based anti-CD33×CD56 bispecific molecule comprising a Fab fragment that binds CD33 and a Fab fragment that binds CD56.

As used herein the term “specific” and “specifically” are used interchangeably to indicate that biomolecules other than CD33, or CD56 (or where the biomolecule is a bispecific molecule both CD33 and CD56) do not significantly bind to the antibody. In some embodiments, the level of binding to a biomolecule other than CD33, or CD56 is negligible (e.g., not determinable) by means of ELISA or an affinity determination.

By “negligible binding” a binding is meant, which is at least about 85%, particularly at least about 90%, more particularly at least about 95%, even more particularly at least about 98%, but especially at least about 99% and up to 100% less than the binding to CD33 or CD56.

The binding affinity of an antibody to a peptide or epitope may be determined with a standard binding assay, such as surface plasmon resonance technique (BIAcore®, GE-Healthcare Uppsala, Sweden). The term “surface plasmon resonance,” as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51: 19-26; Jonsson, U., et al. (1991) Biotechniques 11:620-627; Johnsson, B., et al. (1995) J. Mol. Recognit. 8: 125-131; and Johnnson, B., et al. (1991) Anal. Biochem. 198:268-277.

In one embodiment, the antibody or antigen binding fragments thereof, or cell inhibiting agents, of the invention are capable of mediating antibody dependent cell cytotoxicity. Antibody dependent cellular cytotoxicity (ADCC) is an immune effector cell mediated mechanism which may contribute to anti-tumour activity of monoclonal antibodies (Weiner G J. Monoclonal antibody mechanisms of action in cancer. Immunol Res. 2007, 39(1-3):271-8). The relevance of ADCC for anti-tumour efficacy has been demonstrated in preclinical models, e.g., in mouse tumour models (e.g., Clynes R A, Towers T L, Presta L G, Ravetch J V. Inhibitory Fc receptors modulate in vivo cytotoxicity against tumour targets. Nat Med. 2000 April; 6(4):443-6). Data from clinical trials support the relevance of ADCC for clinical efficacy of therapeutic antibodies (e.g., Weng W K, Levy R Two immunoglobulin G fragment C receptor polymorphisms independently predict response to rituximab in patients with follicular lymphoma. J Clin Oncol. 2003 November I; 21(21):3940-7. Epub 2003 Sep. 15). Interactions of monoclonal antibodies with Fc receptors on immune cells contribute to ADCC. The Fc of antibodies can be modified in order to display enhanced affinity to Fc receptors (e.g., Presta LG Engineering of therapeutic antibodies to minimise immunogenicity and optimise function. Adv Drug Deliv Rev. 2006 Aug. 7; 58(5-6):640-56. Epub 2006 May 23). Such enhanced affinity to Fc receptors results in increased ADCC activity which may lead to increased anti-tumour efficacy in patients.

Thus, in one embodiment, the bispecific antibody comprises an Fc region. Thus, the cell inhibiting agent may be a bispecific antibody that binds CD33 and CD56 and which comprises an Fc region.

In an alternative embodiment, the antigen binding fragments thereof of the invention are immunoresponsive cells which expresses a chimeric antigen T cell receptor protein (CAR), wherein the chimeric T cell receptor protein specifically binds to CD33 and CD56. In one embodiment immunoresponsive cell is bispecific and which a chimeric antigen T cell receptor protein (CAR), wherein the chimeric T cell receptor protein specifically binds to CD33 and a chimeric antigen T cell receptor protein (CAR), wherein the chimeric T cell receptor protein specifically binds to CD56.

In some embodiments, the immunoresponsive cell is autologous to the subject. In another embodiment, the immunoresponsive cell is not autologous to the subject. In a particular embodiment, the immunoresponsive cell is a T cell and is autologous to the subject to be treated.

In some embodiments, the antibody or antigen binding fragments thereof, or agents, e.g. cell inhibiting agents, comprises a binding portion (i.e. a CD33 binding portion and a CD56 binding portion,) and payload, for example a cell killing agent, an immune-modulating payload, a macrophage class switching agent or a light activatable payload. In certain embodiments, the cell binding portion is an antibody or antigen binding fragments thereof. In particular embodiments the cell binding portion is an antibody or antigen binding fragments thereof.

In some embodiments, the antibody or antigen binding fragments thereof, or agents, e.g. cell inhibiting agents, further comprises (or is incorporated or associated with) a cytotoxic or cytostatic agent, i.e., a compound that kills or inhibits tumour cells. Such agents may impart their cytotoxic and cytostatic effects by mechanisms including tubulin binding, DNA binding, proteasome and/or topoisomerase inhibition.

The cytotoxic or cytostatic agent may be, for example, a peptide toxin, a small molecule toxin or a radioisotope. This is also referred to herein as drug or cytotoxic payload.

As used herein, an “ADC” is an antibody drug conjugate.

In one embodiment the cytotoxic or cytostatic agent may be a tubulin inhibitor; or a DNA interacting agent. Tubulin inhibitors modulate tubulin polymerisation. DNA interacting agents target cellular DNA.

In an embodiment the cytotoxic or cytostatic agent is a tubulin inhibitor. In an embodiment, the tubulin inhibitor is selected from the group consisting of: (a) an auristatin; and (b) a maytansine derivative. In an embodiment, the cytotoxic or cytostatic agent is an auristatin. Auristatins include synthetic derivatives of the naturally occurring compound Dolastatin-10. Auristatins are a family of antineoplastic/cytostatic pseudopeptides. Dolastatins are structurally unique due to the incorporation of 4 unusual amino acids (Dolavaine, Dolaisoleuine, Dolaproine and Dolaphenine) identified in the natural biosynthetic product. In addition, this class of natural product has numerous asymmetric centres defined by total synthesis studies by Pettit et al (U.S. Pat. No. 4,978,744). It would appear from structure activity relationships that the Dolaisoleuine and Dolaproine residues appear necessary for antineoplastic activity (U.S. Pat. Nos. 5,635,483 and 5,780,588). In an embodiment, the auristatin is selected from the group consisting of: Auristatin E (AE); Monomethylauristatin E (MMAE); Auristatin F (MMAF); vcMMAE; vcMMAF; mcMMAE and mcMMAF. In an embodiment, the cytotoxic or cytostatic agent is a maytansine or a structural analogue of maytansine. In an embodiment, the cytotoxic or cytostatic agent is a maytansine. Maytansines include structurally complex antimitotic polypeptides. Maytansines are potent inhibitors of microtubulin assembly which leads towards apoptosis of tumour cells. In an embodiment the maytansine is selected from the group consisting of: Mertansine (DM1); and a structural analogue of maytansine such as DM3 or DM4. Preferably, the drug is MMAE, MMAF or auristatin MMAF.

In an embodiment, the cytotoxic or cytostatic agent is DNA interacting agent. In an embodiment, the DNA interacting agent is selected from the group consisting of: (a) calicheamicins, (b) duocarmycins and (c) pyrrolobenzodiazepines (PBDs). In an embodiment, the cytotoxic or cytostatic agent is a calicheamicin. Calicheamicin is a potent cytotoxic agent that causes double-strand DNA breaks, resulting in cell death. Calicheamicin is a naturally occurring enediyne antibiotic (A. L. Smith et al, J. Med. Chem., 1996, 39, 11, 2103-2117). Calicheamicin was found in the soil microorganism Micromonosporaechinospora. In an embodiment, the calicheamicin is calicheamicin gamma 1. In an embodiment, the drug is a duocarmycin. Duocarmycins are potent anti-tumour antibiotics that exert their biological effects through binding sequence-selectively in the minor groove of DNA duplex and alkylating the N3 of adenine (D. Boger, Pure & Appl. Chem., 1994, 66, 4, 837-844). In an embodiment, the duocarmycin is selected from the group consisting of: Duocarmycin A; Duocarmycin 1; Duocarmycin B2; Duocarmycin C1; Duocarmycin C2; Duocarmycin D; Duocarmycin SA; Cyclopropylbenzoindole (CBI) duocarmycin; Centanamycin; Rachelmycin (CC-1065); Adozelesin; Bizelesin; and Carzelesin. In an embodiment, the cytotoxic or cytostatic agent is a pyrrolobenzodiazepine. Pyrrolobenzodiazepines (PBDs) are a class of naturally occurring anti-tumour antibiotics. Pyrrolobenzodiazepines are found in Streptomyces. PBDs exert their anti-tumour activity by covalently binding to the DNA in the minor groove specifically at purine-guanine-purine units. They insert on to the N2 of guanine via an aminal linkage and, due to their shape, they cause minimal disruption to the DNA helix. It is believed that the formation of the DNA-PBD adduct inhibits nucleic acid synthesis and causes excision-dependent single and double stranded breaks in the DNA helix. As synthetic derivatives the joining of two PBD units together via a flexible polymethylene tether allows the PBD dimers to cross-link opposing DNA strands producing highly lethal lesions. In an embodiment, the cytotoxic or cytostatic agent is a synthetic derivative of two pyrrolobenzodiazepines units joined together via a flexible polymethylene tether. In an embodiment, the pyrrolobenzodiazepine is selected from the group consisting of: Anthramycin (and dimers thereof); Mazethramycin (and dimers thereof); Tomaymycin (and dimers thereof); Prothracarcin (and dimers thereof); Chicamycin (and dimers thereof); Neothramycin A (and dimers thereof); Neothramycin B (and dimers thereof); DC-81 (and dimers thereof); Sibiromycin (and dimers thereof); Porothramycin A (and dimers thereof); Porothramycin B (and dimers thereof); Sibanomycin (and dimers thereof); Abbeymycin (and dimers thereof); SG2000; and SG2285.

In an embodiment, the cytotoxic or cytostatic agent is a drug that targets DNA interstrand crosslinks through alkylation. A drug that targets DNA interstrand crosslinks through alkylation is selected from: a DNA targeted mustard; a guanine-specific alkylating agent; and a adenine-specific alkylating agent. In an embodiment, the cytotoxic or cytostatic agent is a DNA targeted mustard. For example, the DNA targeted mustard may be selected from the group consisting of: an oligopyrrole; an oligoimidazole; a Bis-(benzimidazole) carrier; a Polybenzamide Carrier; and a 9-Anilinoacridine-4-carboxamide carrier.

In an embodiment, the cytotoxic or cytostatic agent is selected from the group consisting of: Netropsin; Distamycin; Lexitropsin; Tallimustine; Dibromotallimustine; PNU 157977; and MEN 10710.

In an embodiment, the cytotoxic or cytostatic agent is a Bis-(benzimidazole) carrier. Preferably, the drug is Hoechst 33258.

A guanine-specific alkylating agent is a highly regiospecific alkylating agents that reacts at specific nucleoside positions. In an embodiment, the cytotoxic or cytostatic agent is a guanine-specific alkylating agent selected from the group consisting of: a G-N2 alkylators; a A-N3 alkylator; a mitomycin; a carmethizole analogue; a ecteinascidin analogue. In an embodiment, the mitomycin is selected from: Mitomycin A; Mitomycin C; Porfiromycin; and KW-2149. In an embodiment, the a carmethizole analogue is selected from: Bis-(Hydroxymethyl)pyrrolizidine; and NSC 602668. In an embodiment, the ecteinascidin analogue is Ecteinascidin 743.

Adenine-specific alkylating agents are regiospecific and sequence-specific minor groove alkylators reacting at the N3 of adenines in polypyrimidines sequences.

Cyclopropaindolones and duocamycins may be defined as adenine-specific alkylators. In an embodiment, the cytotoxic or cytostatic agent is a cyclopropaindolone analogue. Preferably, the drug is selected from: adozelesin; and carzelesin.

In an embodiment, the cytotoxic or cytostatic agent is a benz[e]indolone. Preferably, the cytotoxic or cytostatic agent is selected from: CBI-TMI; and iso-CBI.

In an embodiment, the cytotoxic or cytostatic agent is bizelesin. In an embodiment, the cytotoxic or cytostatic agent is a Marine Antitumour Drug. Marine Antitumour Drugs has been a developing field in the antitumour drug development arena (I. Bhatnagar et al, March Drugs 2010, 8, P2702-2720 and T. L. Simmons et al, Mol. Cancer Ther. 2005, 4(2), P333-342). Marine organisms including sponges, sponge-microbe symbiotic association, gorgonian, actinomycetes, and soft coral have been widely explored for potential anticancer agents.

In an embodiment, the cytotoxic or cytostatic agent is selected from: Cytarabine, Ara-C; Trabectedin (ET-743); and EribulinMesylate. In an embodiment, the EribulinMesylate is selected from: (E7389); Soblidotin (TZT 1027); Squalamine lactate; CemadotinPlinabulin (NPI-2358); Plitidepsin; Elisidepsin; Zalypsis; Tasidotin, Synthadotin; (ILX-651); Discodermolide; HT1286; LAF389; Kahalalide F; KRN7000; Bryostatin 1; Hemiasterlin (E7974); Marizomib; Salinosporamide A; NPI-0052); LY355703; CRYPTO 52; Depsipeptide (NSC630176); Ecteinascidin 743; Synthadotin; Kahalalide F; Squalamine; Dehydrodidemnin B; Didemnin B; Cemadotin; Soblidotin; E7389; NVP-LAQ824; Discodermolide; HTI-286; LAF-389; KRN-7000 (Agelasphin derivative); Curacin A; DMMC; Salinosporamide A; Laulimalide; Vitilevuamide; Diazonamide; Eleutherobin; Sarcodictyin; Peloruside A; Salicylihalimides A and B; Thiocoraline; Ascididemin; Variolins; Lamellarin D; Dictyodendrins; ES-285 (Spisulosine); and Halichondrin B.

The following cytotoxic or cytostatic agent are also encompassed by the present invention: Amatoxins (α-amanitin)-bicyclic octapeptides produced by basidiomycetes of the genus Amanita, e.g., the Green Deathcap mushroom; Tubulysins; Cytolysins; dolabellanins; Epothilone A, B, C, D, E, F. Epothilones—constitute a class of non-taxane tubulin polymerisation agents and are obtained by natural fermentation of the myxobacterium Sorangiumcellulosum. These moieties possess potent cytotoxic activity which is linked to the stabilisation of microtubules and results in mitotic arrest at the G2/M transition. Epothilones have demonstrated potent cytotoxicity across a panel of cancer cell lines and has often exhibited greater potency than paclitaxel (X. Pivot et al, European Oncology, 2008; 4(2), P42-45). In an embodiment, the drug is amatoxin. In an embodiment, the drug is tubulysin. In an embodiment, the drug is cytolysin. In an embodiment, the drug is dolabellanin. In an embodiment, the drug is epothilone.

The following cytotoxic or cytostatic agent are also encompassed by the present invention. In an embodiment, the drug is selected from: Doxorubicin; Epirubicin; Esorubicin; Detorubicin; Morpholino-doxorubicin; Methotrexate; Methopterin; Bleomycin; Dichloromethotrexate; 5-Fluorouracil; Cytosine-β-D-arabinofuranoside; Taxol; Anguidine; Melphalan; Vinblastine; Phomopsin A; Ribosome-inactivating proteins (RIPs); Daunorubicin; Vinca alkaloids; Idarubicin; Melphalan; Cis-platin; Ricin; Saporin; Anthracyclines; Indolino-benzodiazepines; 6-Mercaptopurine; Actinomycin; Leurosine; Leurosideine; Carminomycin; Aminopterin; Tallysomycin; Podophyllotoxin; Etoposide; Hairpin polyamides; Etoposide phosphate; Vinblastine; Vincristine; Vindesine; Taxotere retinoic acid; N8-acetyl spermidine; Camptothecin; Esperamicin; and Ene-diynes.

In one embodiment, the cell killing portion is a peptide toxin, for example an auristatin such as MMAE or MMAF. In one embodiment, the antibody or antigen binding fragments thereof, or cell inhibiting agents, comprises a binding portion and a cell killing portion, wherein the binding portion is an anti-CD33 anti-CD56 bispecific antibody or binding portion thereof and wherein the cell killing portion is a peptide toxin, for example an auristatin such as Auristatin E (AE); Monomethylauristatin E (MMAE); Auristatin F (MMAF), vcMMAE, vcMMAF, mcMMAE and mcMMAF.

In certain embodiments, the antibody or antigen binding fragments thereof, or cell inhibiting agents, comprises a binding portion that is conjugated to a payload, for example a cell killing agent, an immune-modulating payload, a macrophage class switching agent or a light activatable payload. Such conjugates may be prepared by in vitro methods known to one of ordinary skill in the art. Techniques for conjugating cytotoxic or cytostatic agent to proteins, and in particular to antibodies, are well-known. (See, e.g., Alley et ah, Current Opinion in Chemical Biology 2010 14: 1-9; Senter, Cancer J., 2008, 14(3): 154-169.)

In certain embodiments, a linking group is used to conjugate the binding portion and the payload, for example a cell killing agent, an immune-modulating payload, a macrophage class switching agent or a light activatable payload.

The linker can be cleavable under intracellular conditions, such that cleavage of the linker releases the payload from the binding portion in the intracellular environment. The cleavable linker can be, e.g., a peptidyl linker that is cleaved by an intracellular peptidase or protease enzyme, including a lysosomal or endosomal protease. Cleaving agents can include cathepsins B and D and plasmin (see, e.g., Dubowchik and Walker, Pharm. Therapeutics 83:67-123, 1999). Most typical are peptidyl linkers that are cleavable by enzymes that are present in NTB-A-expressing cells. For example, a peptidyl linker that is cleavable by the thiol-dependent protease cathepsin-B, which is highly expressed in cancerous tissue, can be used (e.g., a linker comprising a Phe-Leu or a Val-Cit peptide).

The cleavable linker can be pH-sensitive, i.e., sensitive to hydrolysis at certain pH values. Typically, the pH-sensitive linker is hydrolysable under acidic conditions. For example, an acid-labile linker that is hydrolysable in the lysosome (e.g., a hydrazone, semicarbazone, thiosemicarbazone, cis-aconitic amide, orthoester, acetal, ketal, or the like) can be used.

Other linkers are cleavable under reducing conditions (e.g., a disulfide linker). The cleavable linker can also be a malonate linker (Johnson et al, Anticancer Res. 15:1387-93, 1995), a maleimidobenzoyl linker (Lau et al, Bioorg-Med-Chem. 3: 1299-1304, 1995), or a 3′-N-amide analogue (Lau et al, Bioorg-Med-Chem. 3: 1305-12, 1995).

In some embodiments the linker can be a protease cleavable linker, for example a valine-citrulline, which may be cleaved by cathepsin B in the lysosome.

The linker also can be a non-cleavable linker, such as a maleimidoca-proyl (mc) linker or maleimido-alkylene- or maleimide-aryl linker that is directly attached to the therapeutic agent and released by proteolytic degradation of the binding portion.

The terms “conjugation” and “conjugate(d)” refer to chemical linkages, either covalent or non-covalent, which proximally associates one molecule of interest with a second molecule of interest.

The conjugate may be prepared by several routes, employing organic chemistry reactions, conditions, and reagents known to those skilled in the art, including: (1) reaction of a nucleophilic group or an electrophilic group of an antibody with a bivalent linker reagent, to form antibody-linker intermediate Ab-L, via a covalent bond, followed by reaction with an activated drug moiety D; and (2) reaction of a nucleophilic group or an electrophilic group of a drug moiety with a linker reagent, to form drug-linker intermediate D-L, via a covalent bond, followed by reaction with the nucleophilic group or an electrophilic group of an antibody. Conjugation methods (1) and (2) may be employed with a variety of antibodies, drug moieties, and linkers to prepare the antibody-drug conjugates described here.

Several specific examples of methods of preparing bispecific antibodies ADCs are known in the art. Traditional methods such as the hybrid hybridoma and chemical conjugation methods can be used in the preparation of the bispecific antibodies of the invention. Co-expression in a host cell of two antibodies, consisting of different heavy and light chains, leads to a mixture of possible antibody products in addition to the desired bispecific antibody, which can then be isolated by, e.g., affinity chromatography or similar methods.

Strategies favoring the formation of a functional bispecific, product, upon co-expression of different antibody constructs can also be used. Strategies for promoting heterodimerization are known in the art. One strategy to promote formation of heterodimers over homodimers is a “knob-into-hole” strategy in which a protuberance is introduced on a first heavy-chain polypeptide and a corresponding cavity in a second heavy-chain polypeptide, such that the protuberance can be positioned in the cavity at the interface of these two heavy chains so as to promote heterodimer formation and hinder homodimer formation.

Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups. Certain antibodies have reducible interchain disulfides, i.e. cysteine bridges. Antibodies may be made reactive for conjugation with linker reagents by treatment with a reducing agent.

Additional nucleophilic groups can be introduced into antibodies through the reaction of lysines with 2-iminothiolane (Traut's reagent) resulting in conversion of an amine into a thiol.

Antibody-drug conjugates may also be produced by modification of the antibody to introduce electrophilic moieties, which can react with nucleophilic substituents on the linker reagent or drug. The sugars of glycosylated antibodies may be oxidized, e.g. with periodate oxidizing reagents, to form aldehyde or ketone groups which may react with the amine group of linker reagents or drug moieties. The resulting imine Schiff base groups may form a stable linkage, or may be reduced, e.g. by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the protein that can react with appropriate groups on the drug. In another embodiment, proteins containing N-terminal serine or threonine residues can react with sodium meta-periodate, resulting in production of an aldehyde in place of the first amino acid. Such aldehyde can be reacted with a drug moiety or linker nucleophile.

Likewise, nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

In another embodiment, the second moity is a label, for example a fluorescent molecule, β-galactosidase, luciferase molecules, chemical dyes, fluorophores or a radioisotope.

There are several methods by which to produce recombinant antibodies which are known in the art. One of these is production in an E. coli expression system. In this embodiment, nucleic acids encoding the antibody or antigen-binding fragment thereof as described in previous aspects of the invention may be inserted into a plasmid and expressed in a suitable expression system. For example, the present invention includes methods for expressing an antibody or antigen-binding fragment thereof or immunoglobulin chain thereof in a host cell (e.g., bacterial host cell such as E. coli, CHO, HEK or other host cell according to the above described aspects of the invention).

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise. Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The invention also relates to the following aspects

1. A composition for use in the non-immune suppressing treatment of a malignancy, wherein the composition comprises a cell inhibiting agent that binds to CD33 and CD56.

2. The composition for use according to aspect 1, wherein the non-immune suppressing treatment of the malignancy is a non-myelosuppressing treatment.

3. The composition for use according to any preceding aspect, wherein the composition is an antibody or antigen binding fragment thereof.

4. The composition for use according to aspect 3, wherein the composition further comprises a cell killing agent.

5. The composition for use according to aspect 4, wherein the cell killing agent comprises a cytotoxin.

6. The composition for use according to aspect 5, wherein said cytotoxin is selected from:
i) a peptide toxin; or
ii) a chemical toxin.

7. The composition for use according to aspects 4 to 6, wherein the composition further comprises a linker for linking the cell killing agent to the cell inhibiting agent that binds to CD33 and CD56 expressed on the cell surface.

8. The composition for use according to aspects 1 to 7, wherein the composition is a bispecific antibody drug conjugate.

9. The composition for use according to aspects 1 to 8, wherein the malignancy is selected from one of the following cancers: haematological cancers or Multiple Myeloma.

10. The composition for use according to aspects 1 to 9, wherein the malignancy is an AML or AML derived cancer.

11. A combination of cell inhibiting agents for use in the non-immune suppressing treatment of a malignancy, wherein the cell inhibiting agents bind to CD33 and CD56.

12. The combination for use according to aspect 11, the non-immune suppressing treatment is non-myelosuppressing.

13. The combination for use according to any of aspects 11 to 12, wherein the cell inhibiting agents comprise antibodies or antigen binding fragment thereof.

14. The combination for use according to aspect 13, wherein the cell inhibiting agents further comprises a cell killing agent.

15. The combination for use according to aspect 14, wherein the cell killing agent comprises a cytotoxin.

16. The combination for use according to aspect 15, wherein said cytotoxin is selected from:
i) a peptide toxin; or
ii) a chemical toxin.

17. The combination for use according to aspects 14 to 16, wherein the cell inhibiting agents further comprises a linker for linking the cell killing agent to the cell inhibiting agent that binds to CD33 and CD56 expressed on the cell surface.

18. The combination for use according to aspects 11 to 17, wherein the cell inhibiting agent is an antibody drug conjugate.

19. The combination for use according to aspects 11 to 18, wherein the malignancy is selected from one of the following cancers: haematological cancers or Multiple Myeloma.

20. The combination for use according to aspects 11 to 19, wherein the malignancy is an AML or AML derived cancer.

21. A cell inhibiting agent for use in the non-immune suppressing treatment of a malignancy, wherein the cell inhibiting agent bispecifically binds to CD33 and CD56.

22. The cell inhibiting agent for use according to aspect 21, wherein the non-immune suppressing treatment is non-myelosuppressing.

23. The cell inhibiting agent for use according to any one of aspects 21 to 22, wherein the cell inhibiting agent is an antibody or antigen binding fragment thereof.

24. The cell inhibiting agent for use according to aspect 23, wherein the cell inhibiting agent further comprises a cell killing agent.

25. The cell inhibiting agent for use according to aspect 24, wherein the cell killing agent comprises a cytotoxin.

26. The cell inhibiting agent for use according to aspect 25, wherein said cytotoxin is selected from:
i) a peptide toxin; or
ii) a chemical toxin.

27. The cell inhibiting agent for use according to aspects 21 to 26, wherein the cell inhibiting agent is a bispecific antibody drug conjugate.

28. The cell inhibiting agent for use according to aspects 19 to 27, wherein the malignancy is selected from one of the following cancers: haematological cancers or Multiple Myeloma.

29. The cell inhibiting agent for use according to aspects 21 to 28, wherein the malignancy is an AML or AML derived cancer.

### FIGURES

Embodiments of the invention are described below, by way of example only with reference to and as illustrated in the following figures:

FIG. 1 is a collection of bar charts showing the mean signal intensity and percent of cells expressing either a single or dual antigen within the myeloid populations of each AML sample (row, shaded grey). Black bar charts show mean signal intensity and percent single/dual positive cells of the indicated reference cell types present within the healthy PBMC control sample.

FIG. 2 is two bivariate tSNE plots of tSNE1 vs tSNE2 for each sample visualising cell density (lighter colours indicate higher cell density). The nomenclature of each PBMC cell type is marked on both plots and was defined by FlowSOM metaclusters from seven healthy patient samples of PBMC cells for both CD3+ and CD3− cell types. tSNE1 is shown on the x-axis and tSNE2 is shown on the y-axis. Each metacluster was identified by FlowSOM. The approximate location of each FlowSOM-identified cell type is numbered on the tSNE plots and corresponding cell-type nomenclature (according to its antigen expression) is listed.

FIG. 3 is a bivariate tSNE plot of tSNE1 vs tSNE2 for each sample visualising cell density (lighter colours indicate higher cell density). The nomenclature of each BMMC cell type is marked on the plot and was defined by FlowSOM metaclusters from four healthy patient samples of BMMC cells. tSNE1 is shown on the x-axis and tSNE2 is shown on the y-axis. Each metacluster was identified by FlowSOM. The approximate location of each FlowSOM-identified cell type is numbered on the tSNE plot and corresponding cell-type nomenclature (according to its antigen expression) is listed.

FIG. 4A are two bivariate plots (CD3+ and CD3− cell populations) which show each detection event corresponding to a single PBMC cell. The expression of one of either CD33 or CD56 is shown on the y- and x-axis respectively. The plot shows the manual gating for dual positive events (grey shaded area). The gate threshold for non-specific antibody binding was calculated using the known single antigen positive cell types present. FIG. 4B is a graph that shows the percentage of cell detection events falling within the dual positive gate of FIG. 4A for each PBMC cell population identified by FlowSOM for each PBMC sample.

FIG. 5A is a bivariate plot which shows each detection event corresponding to a single BMMC cell. The expression of one of either CD33 or CD56 is shown on the y- and x-axis respectively. The plot shows the manual gating for dual positive events (grey shaded area). The gate threshold for non-specific antibody binding was calculated using the known single antigen positive cell types present. FIG. 5B is a graph that shows the percentage of cell detection events falling within the dual positive gate of FIG. 5A for each BMMC cell population identified by FlowSOM for each BMMC sample.

FIG. 6A are two bivariate plots (CD3+ and CD3− cell populations) which show each detection event corresponding to a single PBMC cell. The expression of one of either CD25 or CD34 is shown on the y- and x-axis respectively. The plot shows the manual gating for dual positive events (grey shaded area). The gate threshold for non-specific antibody binding was calculated using the known single antigen positive cell types present. FIG. 6B is a graph that shows the percentage of cell detection events falling within the dual positive gate of FIG. 6A for each PBMC cell population identified by FlowSOM for each PBMC sample.

FIG. 7A is a bivariate plot which shows each detection event corresponding to a single BMMC cell. The expression of one of either CD25 or CD34 is shown on the y- and x-axis respectively. The plot shows the manual gating for dual positive events (grey shaded area). The gate threshold for non-specific antibody binding was calculated using the known single antigen positive cell types present. FIG. 7B is a graph that shows the percentage of cell detection events falling within the dual positive gate of FIG. 7A for each BMMC cell population identified by FlowSOM for each BMMC sample.

FIG. 8A are two bivariate plots (CD3+ and CD3− cell populations) which show each detection event corresponding to a single PBMC cell. The expression of one of either CD56 or CD7 is shown on the y- and x-axis respectively. The plot shows the manual gating for dual positive events (grey shaded area). The gate threshold for non-specific antibody binding was calculated using the known single antigen positive cell types present. FIG. 8B is a graph that shows the percentage of cell detection events falling within the dual positive gate of FIG. 8A for each PBMC cell population identified by FlowSOM for each PBMC sample.

FIG. 9A is a bivariate plot which shows each detection event corresponding to a single BMMC cell. The expression of one of either CD56 or CD7 is shown on the y- and x-axis respectively. The plot shows the manual gating for dual positive events (grey shaded area). The gate threshold for non-specific antibody binding was calculated using the known single antigen positive cell types present. FIG. 9B is a graph that shows the percentage of cell detection events falling within the dual positive gate of FIG. 9A for each BMMC cell population identified by FlowSOM for each BMMC sample.

FIG. 10A are two bivariate plots (CD3+ and CD3− cell populations) which show each detection event corresponding to a single PBMC cell. The expression of one of either CD56 or CD11c is shown on the y- and x-axis respectively. The plot shows the manual gating for dual positive events (grey shaded area). The gate threshold for non-specific antibody binding was calculated using the known single antigen positive cell types present. FIG. 10B is a graph that shows the percentage of cell detection events falling within the dual positive gate of FIG. 10A for each PBMC cell population identified by FlowSOM for each PBMC sample.

FIG. 11A is a bivariate plot which shows each detection event corresponding to a single BMMC cell. The expression of one of either CD56 or CD11c is shown on the y- and x-axis respectively. The plot shows the manual gating for dual positive events (grey shaded area). The gate threshold for non-specific antibody binding was calculated using the known single antigen positive cell types present. FIG. 11B is a graph that shows the percentage of cell detection events falling within the dual positive gate of FIG. 11A for each BMMC cell population identified by FlowSOM for each BMMC sample.

FIG. 12A are two bivariate plots (CD3+ and CD3− cell populations) which show each detection event corresponding to a single PBMC cell. The expression of one of either CD33 or CD371 is shown on the y- and x-axis respectively. The plot shows the manual gating for dual positive events (grey shaded area). The gate threshold for non-specific antibody binding was calculated using the known single antigen positive cell types present. FIG. 12B is a graph that shows the percentage of cell detection events falling within the dual positive gate of FIG. 12A for each PBMC cell population identified by FlowSOM for each PBMC sample.

FIG. 13A is a bivariate plot which shows each detection event corresponding to a single BMMC cell. The expression of one of either CD33 or CD371 is shown on the y- and x-axis respectively. The plot shows the manual gating for dual positive events (grey shaded area). The gate threshold for non-specific antibody binding was calculated using the known single antigen positive cell types present. FIG. 13B is a graph that shows the percentage of cell detection events falling within the dual positive gate of FIG. 13A for each BMMC cell population identified by FlowSOM for each BMMC sample.

FIG. 14 is a graph that shows the percentage cell survival (cell kill assay) of CD33+/CD56+ KASUMI-3 cells to act as “target cells” versus CD33−/CD56− DND-39 cells acting as a “negative control”. KASUMI-3 cells when incubated with increasing concentrations of a CD33+/CD56+ antibody drug conjugate (BVX020148). The cell kill assay was conducted using a 9-point dose response of directly conjugated BVX020148 on 20,000 KASUMI-3 or DND-39 cells per well. The plates were incubated at 37° C., 5% CO₂ for 96 hours. Following incubation, 10 μL of WST-1 reagent was added per well and the plates read following a further incubation at 37° C., 5% CO₂ for 4 hours. The data for each reading was plotted in GraphPad PRISM and the IC50 values recorded. IC50 in KASUMI-3 was 0.11 nM and in DND-39 was >100 nM. Error bars represent the standard error of the mean of 2 biological repeats (N=2) performed in duplicate.

FIG. 15 is a graph that shows the effect of specific antibodies on the differentiation of healthy CD34+ progenitor cells to myeloid CD33+ cells using a colony forming unit assay. Human CD34+ progenitor cells were suspended in Methocult and IMDM media and tested against 0, 0.01, 0.1, 1, 3 and 10 nM concentrations of BVX020148 and Gemtuzumab+αFab-MMAF. Gemtuzumab was tested at 3 and 10 nM and cell only sample (C) was used as control. Gemtuzumab+αFab-MMAF was used as a positive control representing a CD33 monospecific ADC using a similar cytotoxic linker-payload with similar Drug:Antibody ratio to BVX020148. The cells were incubated at 37° C., 5% CO₂ for 9 days. Following incubation, colony counts were performed, and the data was plotted in Excel. Error bars represent the standard deviation across duplicate wells.

FIG. 16 is a graph that shows the effect of specific antibodies on the differentiation of healthy CD34+ progenitor cells to myeloid CD33+ cells within a colony forming unit assay. Human CD34+ progenitor cells were suspended in Methocult and IMDM media and tested against 0, 0.01, 0.1, 1, 3 and 10 nM concentrations of BVX020148 and Gemtuzumab+αFab-MMAF. Gemtuzumab was tested at 3 and 10 nM and cell only sample (C) was used as control. Gemtuzumab+αFab-MMAF was used as a positive control representing a CD33 monospecific ADC using a similar cytotoxic linker-payload with similar Drug:Antibody ratio to BVX020148. The cells were incubated at 37° C., 5% CO₂ for 14 days. Following incubation, colony counts were performed, and the data was plotted in Excel. Error bars represent the standard deviation across duplicate wells.

FIG. 17 is a graph that shows the effect of specific antibodies on the differentiation of healthy CD34+ progenitor cells to myeloid CD33+ cells within a colony forming unit assay. Human 34+ progenitor cells were suspended in Methocult and IMDM media and tested against 0, 0.01, 0.1, 1, 3 and 10 nM concentrations of BVX020148 and Gemtuzumab+αFab-MMAF. Gemtuzumab was tested at 3 and 10 nM and cell only sample (C) was used as control. Gemtuzumab+αFab-MMAF was used as a positive control representing a CD33 monospecific ADC using a similar cytotoxic linker-payload with similar Drug:Antibody ratio to BVX020148. The cells were incubated at 37° C., 5% CO₂ for 9 days. Following incubation, colony counts were performed, and the data was plotted in Excel. Error bars represent the standard deviation across duplicate wells.

FIG. 18 is a graph that shows the effect of specific antibodies on the differentiation of healthy CD34+ progenitor cells to myeloid CD33+ cells within a colony forming unit assay. Human 34+ progenitor cells were suspended in Methocult and IMDM media and tested against 0, 0.01, 0.1, 1, 3 and 10 nM concentrations of BVX020148 and Gemtuzumab+αFab-MMAF. Gemtuzumab was tested at 3 and 10 nM and cell only sample (C) was used as control. Gemtuzumab+αFab-MMAF was used as a positive control representing a CD33 monospecific ADC using a similar cytotoxic linker-payload with similar Drug:Antibody ratio to BVX020148. The cells were incubated at 37° C., 5% CO₂ for 14 days. Following incubation, colony counts were performed, and the data was plotted in Excel. Error bars represent the standard deviation across duplicate wells.

FIG. 19. Cell kill assay conducted using a 9-point dose response of directly conjugated BVX04-a0094-AB4A-1 on KASUMI-3, KE-37. SET-2 or DND-39 cells. The plates were incubated at 37° C., 5% CO₂ for 96 hours. Following incubation, 10 μl of WST-1 reagent was added per well and the plates read following a further incubation at 37° C., 5% CO₂ for 3 hours. The data for each reading was plotted in GraphPad PRISM and the IC50 values recorded. IC₅₀ in KASUMI-3 was 0.06 nM and in KE-37, SET-2 and DND-39 was >30 nM. Error bars represent the standard error of the mean of 2 biological repeats (n=2) performed in duplicate.

FIG. 20. Effect of specific antibodies on the differentiation of healthy CD34+ progenitor cells to myeloid CD33+ cells within a colony forming unit assay. Human CD34+ progenitor cells were suspended in Methocult and IMDM media and tested against 0, 1 and 10 nM concentrations of BVX04-a0094-AB4A-1. The cells were incubated at 37° C., 5% CO₂ for 10 days. Following incubation, colony counts were performed, and the data was plotted in Excel. Average±SEM % colony counts relative to control cells only wells. n=2 biological repeats (2 technical replicates per biological repeat)

FIG. 21. Cell kill assay conducted using a 9-point dose response of directly conjugated BVX04-b0097-AB6A-1 KASUMI-3, KE-37. SET-2 or DND-39 cells. The plates were incubated at 37° C., 5% CO₂ for 96 hours. Following incubation, 10 μl of WST-1 reagent was added per well and the plates read following a further incubation at 37° C., 5% CO₂ for 3 hours. The data for each reading was plotted in GraphPad PRISM and the IC50 values recorded. IC₅₀ in KASUMI-3 was 0.06 nM and in KE-37, SET-2 and DND-39 was >30 nM. Error bars represent the standard error of the mean of 2 biological repeats (n=2) performed in duplicate.

FIG. 22. Effect of specific antibodies on the differentiation of healthy CD34⁺ progenitor cells to myeloid CD33⁺ cells within a colony forming unit assay. Human CD34⁺ progenitor cells were suspended in Methocult and IMDM media and tested against 0 and 1 nM concentrations of BVX04-b0097-AB6A-1. The cells were incubated at 37° C., 5% CO₂ for 10 days. Following incubation, colony counts were performed, and the data was plotted in Excel. Average±SD % colony counts relative to control cell only wells. n=1 biological repeats (2 technical replicates per biological repeat).

### EXAMPLES

The invention is further illustrated in the following non-limiting examples.

### Example 1: Phenotypic Analysis of 25 Acute Myeloid Leukaemia (AML) Samples by Mass Cytometry to Evaluate CD33 and CD56 Antigen Co-Expression

A group of 25 patient AML cell samples underwent a process to determine the cell surface expression of CD33 and CD56 on each patient AML cell.

### Methods

AML bone marrow aspirate samples were collected from 30 patients which were split into 4 batches for staining and running on the CyTOF. Batches were pooled together prior to staining using TeMal metal barcoding. A replicate PBMC sample control was included in each batch to confirm similarity in staining efficiencies between batches.

All centrifuge steps are performed at 500rcf/5 mins/4° C. unless stated otherwise. Cryopreserved AML samples were thawed and mixed with 1 mL of 37° C. culture media containing RPMI buffer (Sigma Aldrich; Cat #R0883) containing 10% FCS, L-Glut and penicillin/streptomycin. An additional 8 mL of 37° C. culture media was then added while agitating and cells were immediately centrifuged at room temperature (RT). Samples were resuspended in 5 mL of culture media and then counted using a haemocytometer. 3×10⁶ cells were removed into a separate tube for each sample to be stained for mass cytometry. These samples were washed once in ice cold MaxPAR PBS (Fluidigm; Cat #201058) and then resuspended in the appropriate TeMal barcode and incubated at RT for 10 mins. Cells were then washed twice in cell staining buffer [CSB; Fluidigm, Cat #201068] before being pooled into one tube in PBS. Pooled AML samples were resuspended at 1×10⁷ cells/mL in a working solution (1:1000 dilution in RT MaxPAR PBS) of Cell-ID Cisplatin. Cells were left at RT for 5 mins, after which, 3× volume of CSB was added to each sample before being centrifuged. Cell pellets were then resuspended in FcX blocking solution at 50 μL/3×10⁶ cells (FcX stock diluted 1:10 dilution in CSB) and incubated at room temperature for 10 mins. A 2× concentrated antibody cocktail was then directly added to the cells suspended in FcX solution and incubated for a further 30 mins, agitating after 15 mins. Samples were washed twice in ice cold CSB, once in ice cold PBS before being resuspended in RT 1.6% formaldehyde (1:10 dilution in MaxPAR PBS; Thermo Scientific, Cat #28906) and incubated at RT for 10 mins. Cells were then centrifuged at 800rcf/5 mins/4° C. and resuspended at 3×10⁶ cells/mL in Intercalator solution (1:2000 dilution of 125 nM Cell-ID Intercalator-Ir [Fluidigm, Cat #201192A] in Fix and Perm buffer [Fluidigm, Cat #201067]). Cells were left overnight at 4° C.

The next day, cells were washed once in CSB and twice in CAS solution (Fluidigm, Cat #201240). Cells were filtered through a 30 μM filter-top test tube (Fisher Scientific, Cat #08-771-23), counted, and resuspended at 7.5×10⁵ cells/mL. EQ™ Four Element Calibration Beads were added 1:10 and the sample was run through a Helios mass cytometer, collecting 1 million events for each BMMC sample or 3 million events of the pooled PBMC.

### Data Analysis

Using the CyTOF software, events were normalised against the signal on the EQ™ Four Element Calibration Beads and debarcoded to separate each sample within the TeMal barcode pool. Normalised, individual sample FCS files were then uploaded to Cytobank cloud software for analysis. After sample cleanup (removal of doublets and dead cells) 5 AML samples were removed from analysis due to poor viability (less than 20%). The remaining 25 AML samples plus the batch control PBMC samples were clustered using FlowSOM, which groups cells together according to their similarities in antigen expression (termed metaclusters). FlowSOM metaclusters identified were given a cell type description according to their median antigen phenotypes.

### Results

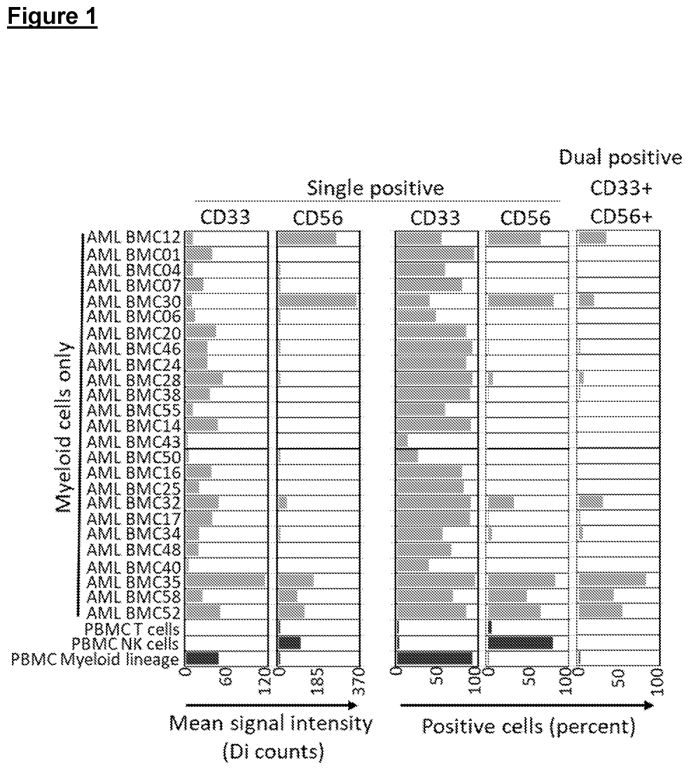

The bar charts of FIG. 1 show that both CD33 and CD56 are expressed on a number of malignant AML cell types isolated from patients with a confirmed haematological malignancy (AML). FIG. 1 shows that in certain patients the majority of malignant myeloid cells express both CD33 and CD56.

The large range of antigens other than CD33 and CD56 that are also expressed on malignant myeloid cells indicates that there is a subset of antigens that are expressed on both the malignant myeloid cells and healthy haematological cells. Targeting antigens expressed on the cell surface of both malignant and healthy myeloid cells would undoubtedly cause malignant cell death however it would also lead to unacceptable targeting of healthy haematological cells.

In order to determine whether CD33 and CD56 may be targeted for the treatment of a haematological malignancy such as AML without causing immune suppression and/or myelosuppression in a patient it is important to determine whether the antigen pair is also expressed on the cell surface of healthy haematological cells.

### Example 2: Multivariate Analysis of 7 Healthy PBMC and 4 Healthy BMMC Patient Samples Characterises Sample Heterogeneity and Identifies Cell Types

Healthy PBMC samples collected from healthy human subjects first underwent a process to separate cells contained in the PBMC samples into cell populations which express CD3 and those which do not. CD3 is almost exclusively found expressed on the cell surface of T-cells, therefore this method separates any T-cells from other peripheral blood mononuclear cells present in the sample.

### Methods

Healthy peripheral blood mononuclear cell (PBMC; acquired from Stem cell, Cat #70025.1) samples derived from 7 separate donors were barcoded and pooled prior to staining (as detailed below), while the healthy bone marrow mononuclear cells (BMMC; acquired from Stem cell, Cat #70001.1) acquired from 4 separate donors were stained and run through a mass cytometer individually.

All centrifuge steps were performed at 500 rcf/5 mins/4° C. unless stated otherwise. 1 mL of cryopreserved cells were thawed and mixed with 1 mL of 37° C. culture media containing RPMI buffer (Sigma Aldrich; Cat #R0883) containing 10% FCS, L-Glut and penicillin/streptomycin. An additional 8 mL of 37° C. culture media was then added while agitating and cells were immediately centrifuged at room temperature (RT). Samples were resuspended in 5 mL of culture media and counted using a haemocytometer. 3×106 cells were removed and placed into a separate tube for each sample to be stained for mass cytometry. These samples were washed once in ice cold MaxPAR PBS (Fluidigm; Cat #201058) and then PBMCs were resuspended in the appropriate anti-CD45 live-cell barcoding mixture (antibodies were diluted in ice cold cell staining buffer [CSB; Fluidigm, Cat #201068]; see Table 1 for antibody details and Table 2 for barcoding strategy), while BMMC were resuspended immediately in Cell-ID Cisplatin (see below). PBMC samples were barcoded on ice for 30 mins before being washed twice in ice cold CSB and washed once in ice cold PBS. During the PBS wash, samples were pooled into one tube before being centrifuged. BMMC or pooled PBMC were resuspended at 107 cells/mL in a working solution (1:1000 dilution in RT MaxPAR PBS) of Cell-ID Cisplatin. Cells were left at RT for 5 mins, after which, 3× volume of CSB was added to each sample before being centrifuged. Cell pellets were then resuspended in FcX blocking solution at 50 μL/3×106 cells (FcX stock diluted 1:10 dilution in CSB) and incubated at room temperature for 10 mins. A 2× concentrated antibody cocktail (see Table 2 for antibody details) was then directly added to the cells suspended in FcX solution and incubated for a further 30 mins, agitating after 15 mins. Samples were washed twice in ice cold CSB, once in ice cold PBS before being resuspended in RT 1.6% formaldehyde (1:10 dilution in MaxPAR PBS; Thermo Scientific, Cat #28906) and incubated at RT for 10 mins. Cells were then centrifuged at 800 rcf/5 mins/4° C. and resuspended at 3×106 cells/mL in Intercalator solution (1:2000 dilution of 125 nM Cell-ID Intercalator-Ir [Fluidigm, Cat #201192A] in Fix and Perm buffer [Fluidigm, Cat #201067]). Cells were left overnight at 4° C. The next day, cells were washed once in CSB and twice in CAS solution (Fluidigm, Cat #201240). Cells were filtered through a 30 μM filter-top test tube (Fisher Scientific, Cat #08-771-23), counted, and resuspended at 7.5×10⁵ cells/mL. EQ™ Four Element Calibration Beads were added 1:10 and the sample was run through a Helios mass cytometer, collecting 1 million events for each BMMC sample or 3 million events of the pooled PBMC.

TABLE 1
Mass cytometry antibody Panel

| | Antigen | | | |
|---|---|---|---|---|
| Metal | Panel | Assay Function | Company | Cat# |
| 89Y | CD45 | Barcoding/Phenotyping | Fluidigm | 3089003B |
| 106Cd | CD45 | Barcoding for PBMC | Biolegend | 304045 |
| 110Cd | CD45 | Barcoding for PBMC | Biolegend | 304045 |
| 111Cd | CD45 | Barcoding for PBMC | Biolegend | 304045 |
| 114Cd | CD45 | Barcoding for PBMC | Biolegend | 304045 |
| 115In | CD371 | Phenotyping | Biolegend | 353602 |
| 116Cd | CD45 | Barcoding for PBMC | Biolegend | 304045 |
| 141Pr | CCR6 | Phenotyping | Fluidigm | 3141014A |
| 142Nd | CD19 | Phenotyping | Fluidigm | 3142001B |
| 143Nd | CD127 | Phenotyping | Fluidigm | 3143012B |
| 144Nd | CD15 | Phenotyping | Fluidigm | 3144019B |
| 145Nd | TIM3 | Phenotyping | Biolegend | 345019 |
| 146Nd | CD7 | Phenotyping | Biolegend | 343111 |
| 147Sm | CD11c | Phenotyping | Fluidigm | 3147008B |
| 148Nd | CD16 | Phenotyping | Fluidigm | 3148004B |
| 149Sm | CCR4 | Phenotyping | Fluidigm | 3149029A |
| 150Nd | CD138 | Phenotyping | Fluidigm | 3150012B |
| 151Eu | CD123 | Phenotyping | Fluidigm | 3151001B |
| 152Sm | CD5 | Phenotyping | Biolegend | 300627 |
| 153Eu | CD33 | Phenotyping | Biolegend | 303419 |
| 154Sm | CD28 | Phenotyping | Biolegend | 302937 |
| 155Gd | CD45RA | Phenotyping | Fluidigm | 3155011B |
| 156Gd | CD10 | Phenotyping | Fluidigm | 3156001B |
| 158Gd | CD135 | Phenotyping | Fluidigm | 3158019B |
| 159Tb | CD96v2 | Phenotyping | Biolegend | 338402 |
| 160Gd | CD13 | Phenotyping | Fluidigm | 3160014B |
| 161Dy | CD90 | Phenotyping | Fluidigm | 3161009B |
| 162Dy | CD66b | Phenotyping | Fluidigm | 3162023B |
| 163Dy | CD34 | Phenotyping | Fluidigm | 3163014B |
| 164Dy | CD49f | Phenotyping | Fluidigm | 3164006B |
| 165Ho | CD45RO | Phenotyping | Fluidigm | 3165011B |
| 166Er | CD24 | Phenotyping | Fluidigm | 3166007B |
| 167Er | CCR7 | Phenotyping | Fluidigm | 3167009A |
| 168Er | CD8 | Phenotyping | Fluidigm | 3168002B |
| 169Tm | CD25 | Phenotyping | Fluidigm | 3169003B |
| 170Er | CD3 | Phenotyping | Fluidigm | 3170001B |
| 171Yb | B7H4 | Phenotyping | Biolegend | 358102 |
| 172Yb | CD38 | Phenotyping | Fluidigm | 3172007B |
| 173Yb | HLADR | Phenotyping | Fluidigm | 3173005B |
| 174Yb | CD4 | Phenotyping | Fluidigm | 3174004B |
| 175Lu | CD14 | Phenotyping | Fluidigm | 3175015B |
| 176Yb | CD56 | Phenotyping | Fluidigm | 3176008B |
| 209Bi | CD70 | Phenotyping | Biolegend | 355102 |

TABLE 2
5-choose-2 Barcoding strategy for PBMC.

| Sample | Barcode | 89 | 106 | 110 | 111 | 114 | 116 |
|---|---|---|---|---|---|---|---|
| PBMC1 | 1 | 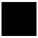 | 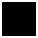 | | | | |
| PBMC2 | 2 | 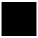 | | 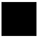 | | | |
| PBMC3 | 3 | 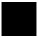 | | | 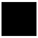 | | |
| PBMC4 | 4 | 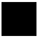 | | | | 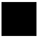 | |
| PBMC5 | 5 | 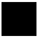 | | | | | 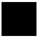 |
| PBMC6 | 6 | | 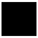 | 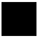 | | | |
| PBMC7 | 7 | | 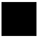 | | 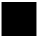 | | |

Samples were initially stained with 2 of the 5 possible metal-isotope conjugated anti-CD45 antibodies shown in Table 2 above, to produce a unique metal combination tag (or barcode) for each sample. Samples could then be pooled together into the same tube allowing them to be stained and run through the mass cytometer simultaneously, greatly reducing technical variability within the experiment. Events could then be separated in silico after data collection into the samples that they originated from by bivariate gating on their barcode metal signal.

### Data Analysis

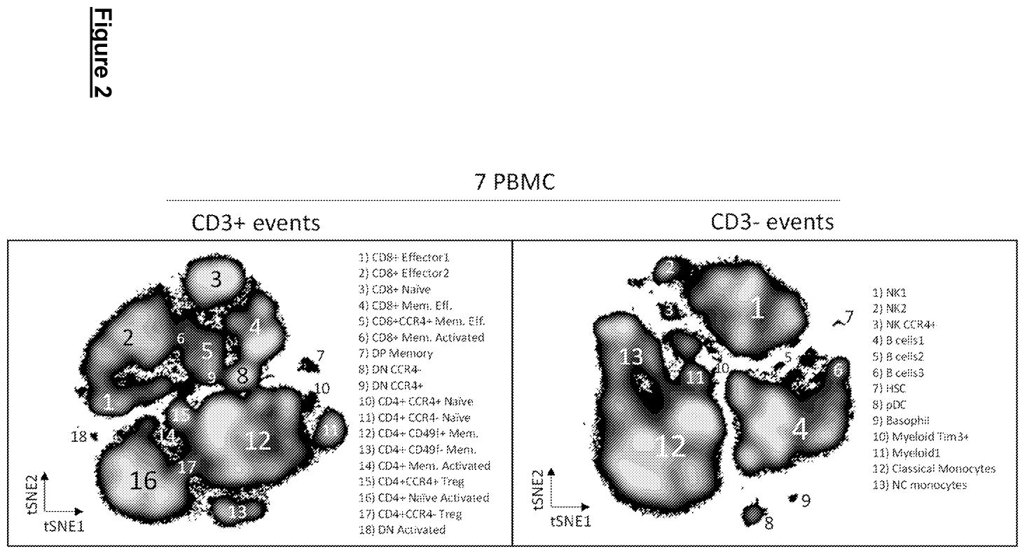

Events were normalised over time against the signal on the EQ™ Four Element Calibration Beads using the CyTOF software. Normalised FCS files were then uploaded to Cytobank cloud software for analysis. For PBMC, samples were debarcoded by separating samples using bivariate Boolean gating of the barcoding metals. Cells from PBMC were also split into CD3− and CD3+ events prior to further multivariate analysis. Cells from each sample type (i.e., CD3+ PBMC, CD3− PBMC, BMMC) were clustered separately using FlowSOM, which groups cells together according to their similarities in antigen expression (termed metaclusters). After FlowSOM analysis, cells from each sample type were also passed through the viSNE algorithm that allows visualisation of the 37 phenotyping antigen dimensions in 2-dimensional space (i.e., tSNE1 and tSNE2); where the values of the 2 new tSNE parameters for each cell is equivalent to its phenotype in multidimensional space. FlowSOM metaclusters identified by FlowSOM could then be overlayed onto the tSNE plot for visualisation and given nomenclature for cell type (e.g., CD4 T cells) according to their median antigen phenotypes (FIG. 2). Cells for each cell type (i.e., FlowSOM metacluster) within each sample type were then analysed for percentage of dual antigen positivity identified using manual bivariate gating.

### Results

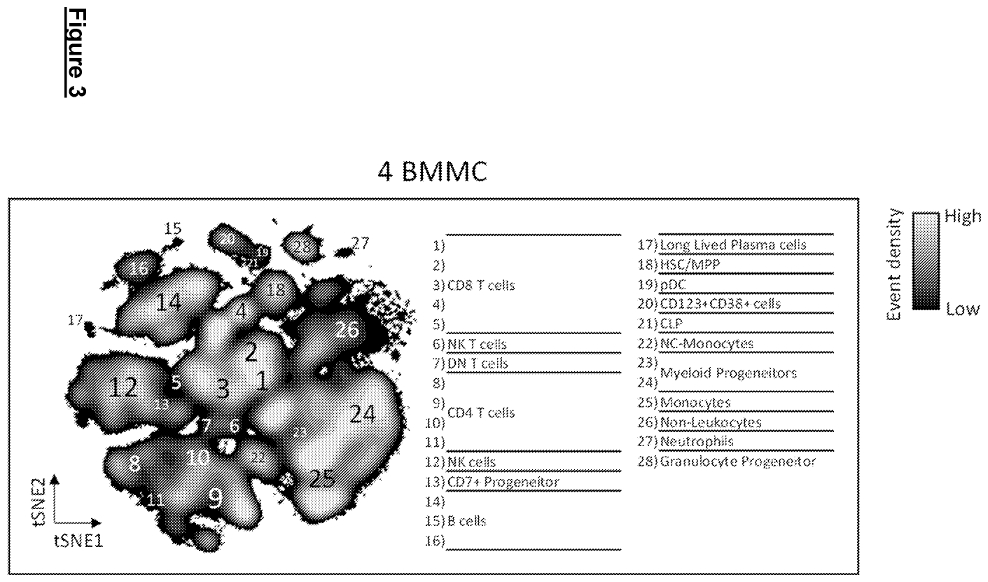

The tSNE plots of FIG. 2 show that a number of different PBMC cell types were identified by FlowSOM. Each of these cell types was identified using the cell surface expression of the specific marker proteins shown in Table 1. Each of the cell surface marker proteins identified is shown on the cell type lists of FIG. 2. For example, the cell population labelled “3” on the CD3+ tSNE plot corresponds to naïve T-cells expressing CD8 on the cell surface; the cell population labelled “1” on the CD3− tSNE plot corresponds to NK1 cells. The tSNE plots of FIG. 3 show that a number of different BMMC cell types were identified by FlowSOM. Each of the cell types was identified using one of the cell surface proteins as shown in Table 1. For example, the cell population labelled “15” on the BMMC tSNE plot corresponds to B cells present within the BMMC patient samples.

By separating the different PBMC and BMMC cell types using this data analysis method it was possible to investigate the overall expression pattern of specific cell surface proteins of each cell type.

### Example 3: Dual CD33 and CD56 Cell Surface Expression on PBMC and BMMC Cell Populations

### Methods

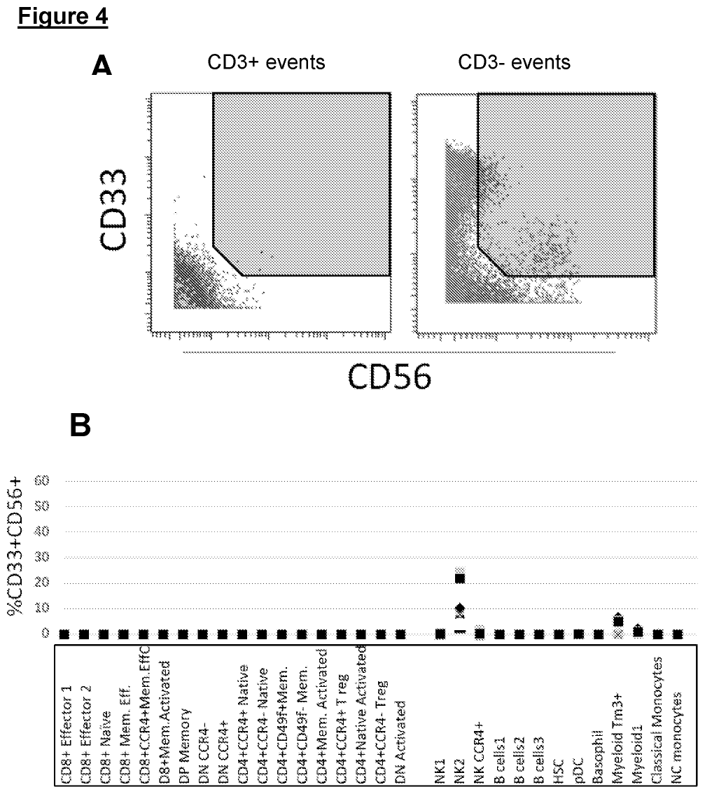

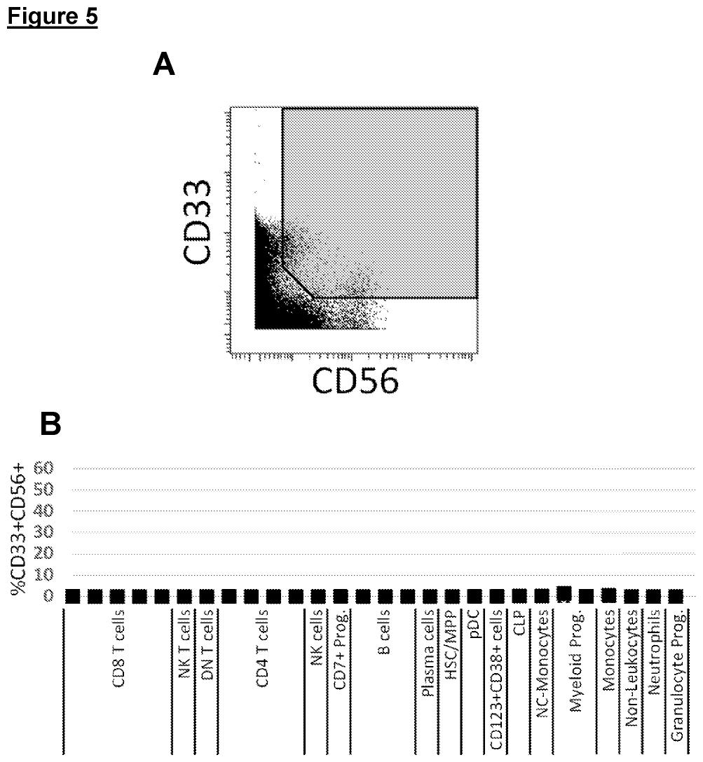

PBMC cell types identified in Example 2 were evaluated for the percentage of dual antigen positive cells. Bivariate plots (FIG. 4A) for both CD3+ and CD3− PBMC cell types were manually gated for positive events matching CD33 and CD56 expression. The threshold for non-specific antibody binding was calculated using the known single antigen positive cell types present in the sample. A percentage within this gate for each cell type identified by FlowSOM in Example 2 above was then calculated and plotted on a graph shown in FIG. 4B. The same method was used to assess the expression pattern of BMMC cells as shown in FIG. 5A and FIG. 5B

The method used to assess the expression pattern of CD33 and CD56 on the cell surface of both PBMC and BMMC cell populations was the same as that used in Example 2 above but with bivariate plots produced for CD33 and CD56 on y- and x-axis respectively (FIG. 4 and FIG. 5).

### Results

FIG. 4A shows that there were few dual CD33+/CD56+ events in both T-cell (CD3+) and non-T-cell (CD3−) PBMC cell types. FIG. 4B shows the percentage of each PBMC cell type population positive for both CD33 and CD56. None of the PBMC cell populations contained any cells that expressed both CD33 and CD56 on their cell surface. FIG. 5A shows that there were very few dual CD33+/CD56+ detection events on BMMC cells from healthy patient samples.

These results show that bispecifically targeting CD33 and CD56 for the treatment of malignancies would effectively target the cancerous cells expressing both CD33 and CD56 while avoiding targeting other healthy haematological cell populations. Using this treatment it would be possible to avoid any off-target cytotoxic effects such as immune suppression and myelosuppression that would otherwise occur when treating malignancies by targeting a single antigen or two antigens expressed on the surface of the same healthy cell.

### Example 4: Dual CD25 and CD34 Cell Surface Expression on PBMC and BMMC Cell Populations

### Methods

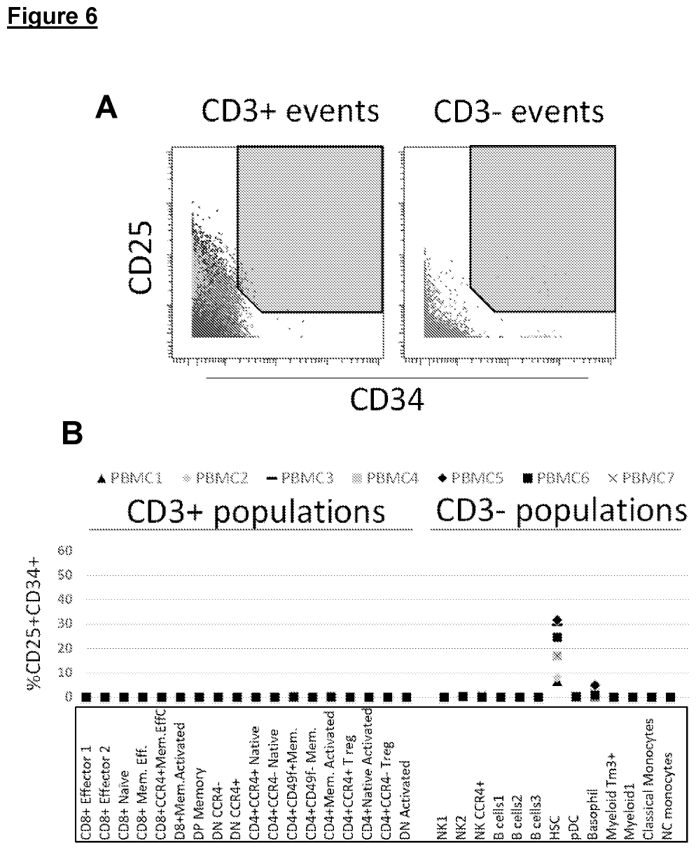

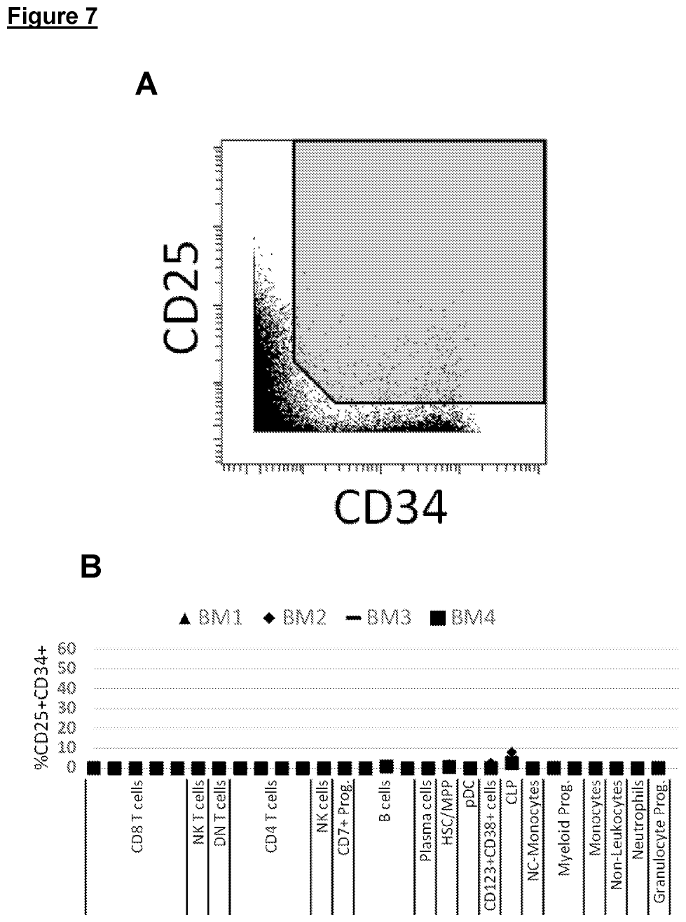

The method used to assess the expression pattern of CD25 and CD34 on the cell surface of both PBMC and BMMC cell populations was the same as that used in Example 3 above but with bivariate plots produced for CD25 and CD34 on y- and x-axis respectively (FIG. 6 and FIG. 7).

### Results

FIG. 4A shows that there were dual CD25+/CD34+ events in the non-T-cell (CD3−) PBMC cell types. FIG. 6B shows the percentage of each PBMC cell type population positive for both CD25 and CD34. FIG. 6B shows that a high percentage of the haematopoietic stem cell (HSC) population identified in Example 2 demonstrate dual expression of both CD25 and CD34. Therefore, any composition that targets both CD25 and CD34 as a treatment for a malignancy would also target HSCs expressing CD25 and CD34. Off-target toxicity to HSCs caused by a treatment targeting CD25 and CD34 would lead to direct myelosuppression in bone marrow, an unwanted and potentially life-threatening side effect of treatment. FIG. 7A shows that there were few dual CD25+/CD34+ detection events on BMMC cells from healthy patient samples. Despite this, any treatment that targets both CD25 and CD34 does not avoid negative off-target cytotoxicity and would lead to myelosuppression by simultaneously targeting HSCs and any malignancy.

### Example 4: Dual CD56 and CD7 Cell Surface Expression on PBMC and BMMC Cell Populations

### Methods

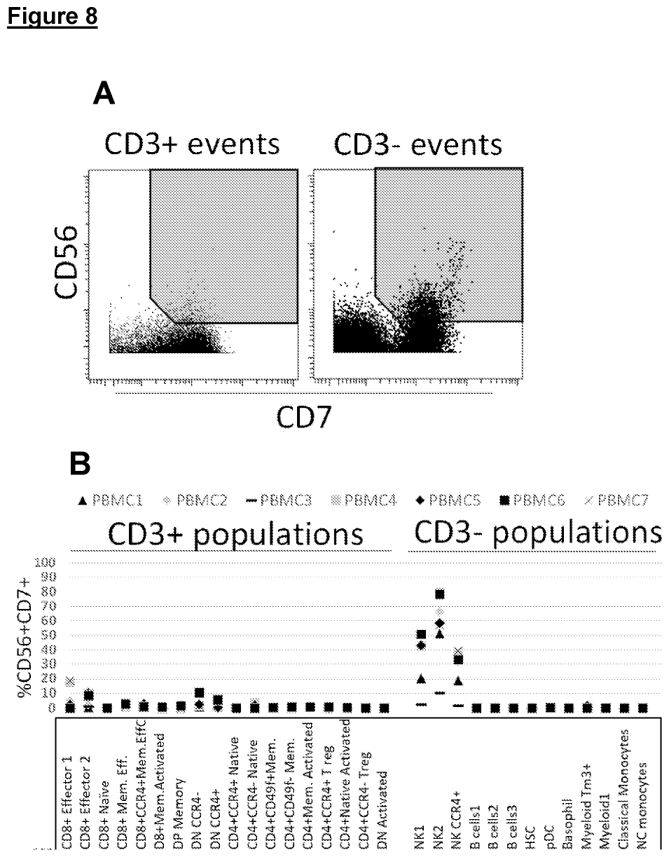

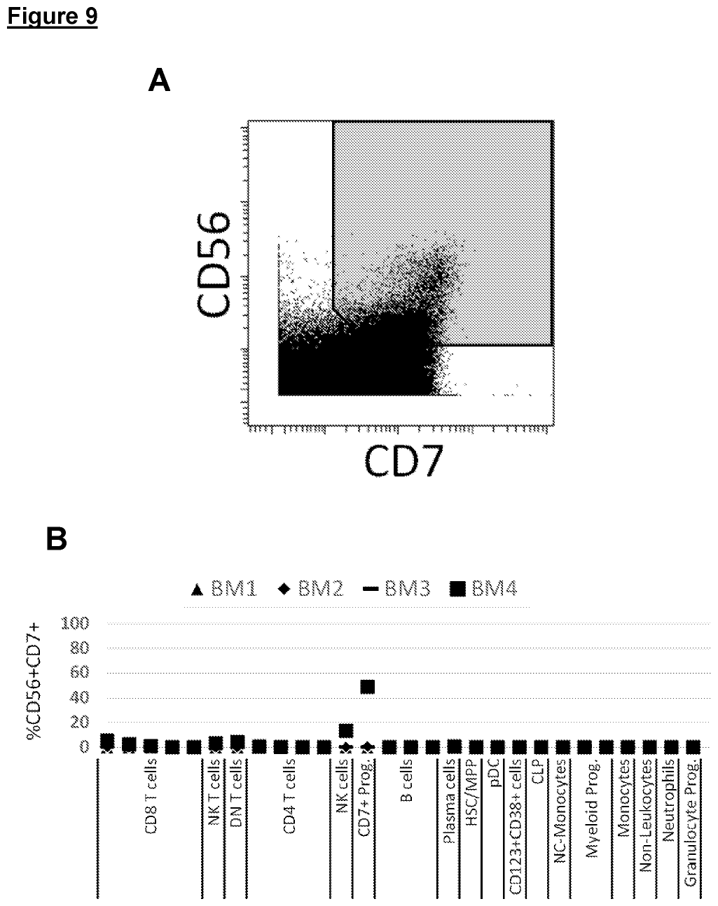

The method used to assess the expression pattern of CD56 and CD7 on the cell surface of both PBMC and BMMC cell populations was the same as that used in Example 3 above but with bivariate plots produced for CD56 and CD7 on y- and x-axis respectively (FIG. 8 and FIG. 9).

### Results

FIG. 8A shows that overall there were a substantial number of dual CD56+/CD7+ events in non-T-cell (CD3−) PBMC cell types. FIG. 8B shows the percentage of each PBMC cell type population positive for both CD56 and CD7. FIG. 8B shows that a high percentage of the Natural Killer 1 (NK1), Natural Killer 2 (NK2) and Natural Killer CCR4+ (NK CCR4+) cell populations identified in Example 2 demonstrate dual expression of both CD56 and CD7 and would therefore experience off-target cytotoxicity caused by a treatment targeting both CD56 and CD7. Targeting NK cells would lead to reduced innate immunity thereby making the patient susceptible to infection and disease. Furthermore, FIG. 8A shows that there were a substantial number of dual CD56+/CD7+ detection events in BMMC cell populations from healthy patient samples. FIG. 8B shows that the majority of the detection events arise from the CD7+ Progenitor cell type. Any off-target cytotoxicity directed towards this cell type leads to immune suppression and potential myelosuppression. Therefore, any treatment that targets both CD56 and CD7 does not avoid negative off-target cytotoxicity and would lead to immune suppression by simultaneously targeting NK cells and BMMCs along with any malignancy.

### Example 5: Dual CD56 and CD11c Cell Surface Expression on PBMC and BMMC Cell Populations

### Methods

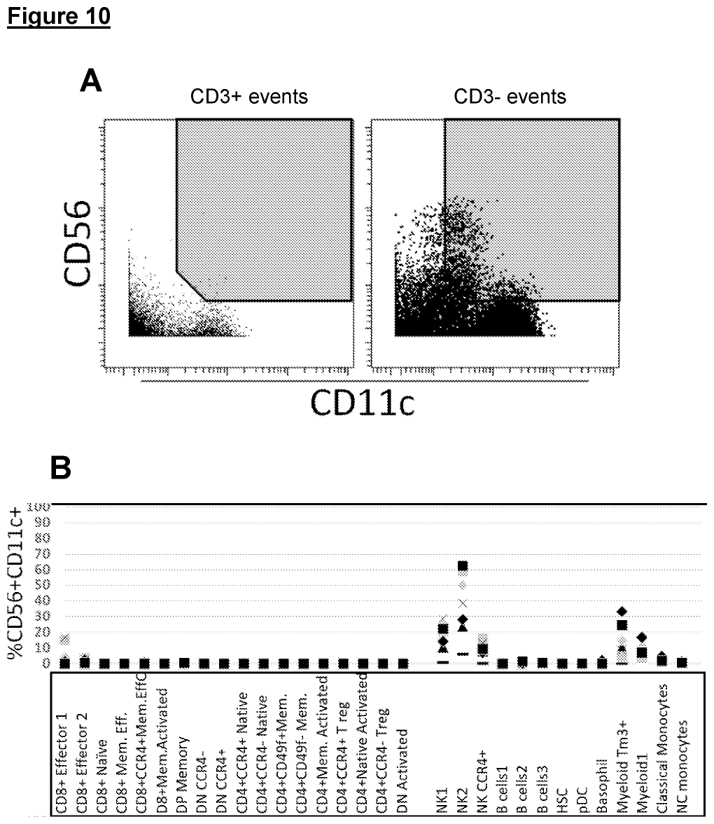

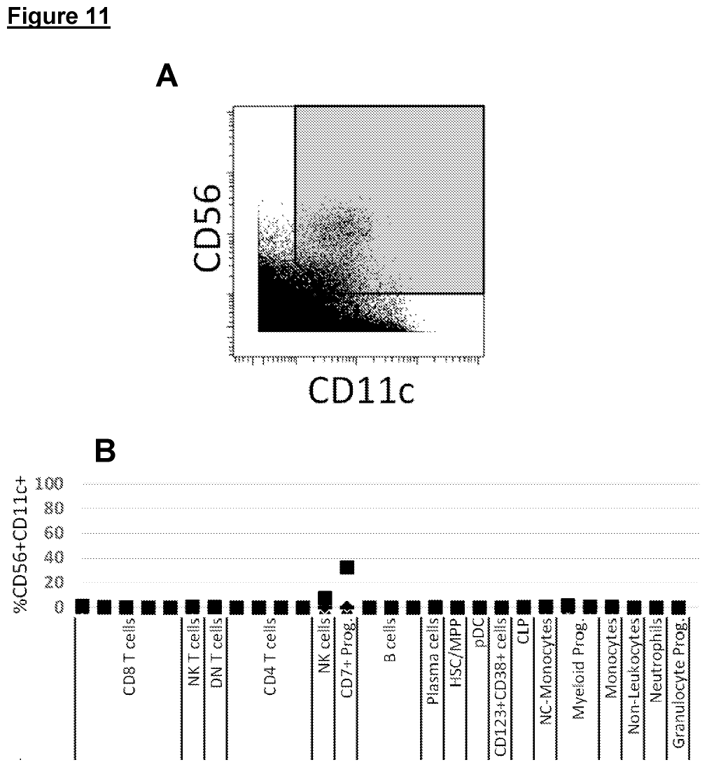

The method used to assess the expression pattern of CD56 and CD11c on the cell surface of both PBMC and BMMC cell populations was the same as that used in Example 3 above but with bivariate plots produced for CD56 and CD11c on y- and x-axis respectively (FIG. 10 and FIG. 11).

### Results

FIG. 10A shows that overall there were a substantial number of dual CD56+/CD11 c+ events in non-T-cell (CD3−) PBMC cell types. FIG. 10B shows the percentage of each PBMC cell type population positive for both CD56 and CD11c. FIG. 10B shows that a high percentage of the Natural Killer 1 (NK1), Natural Killer 2 (NK2) and Natural Killer CCR4+ (NK CCR4+), Myeloid Tlm3 and Myeloid 1 cell populations identified in Example 2 demonstrate dual expression of both CD56 and CD11c and would therefore experience off-target cytotoxicity caused by a treatment targeting both CD56 and CD11c. Targeting NK cells would lead to reduced innate immunity thereby making the patient susceptible to infection and disease. Targeting Myeloid cell populations would lead to myelosuppression. Furthermore, FIG. 11A shows that there were a substantial number of dual CD56+/CD11c+ detection events in BMMC cell populations from healthy patient samples. FIG. 11B shows that the majority of the detection events arise from the CD7+ Progenitor cell type. Any off-target cytotoxicity directed towards this cell type leads to immune suppression and potential myelosuppression. Therefore, any treatment that targets both CD56 and CD11c does not avoid negative off-target cytotoxicity and would lead to immune suppression by simultaneously targeting NK cells, myeloid cells and BMMCs along with any malignancy.

### Example 6: Dual CD33 and CD371 Cell Surface Expression on PBMC and BMMC Cell Populations

### Methods

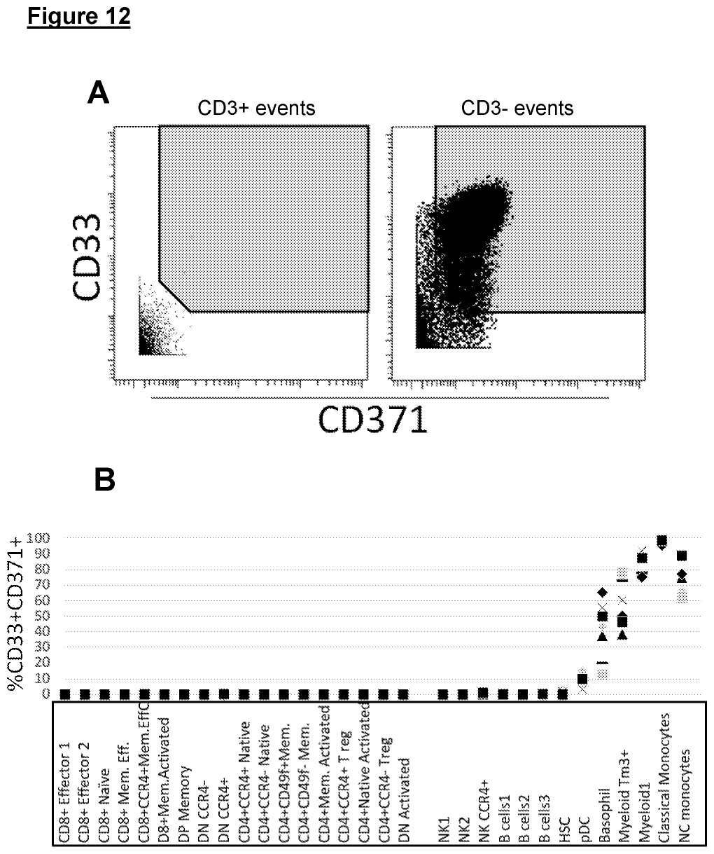

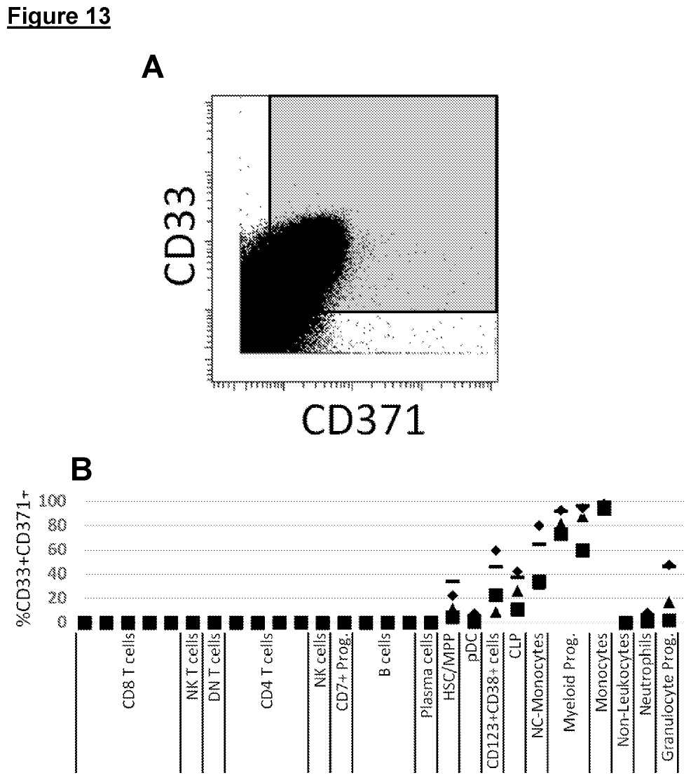

The method used to assess the expression pattern of CD33 and CD371 on the cell surface of both PBMC and BMMC cell populations was the same as that used in Example 3 above but with bivariate plots produced for CD33 and CD371 on y- and x-axis respectively (FIG. 12 and FIG. 13).

### Results

FIG. 12A shows that overall there were a substantial number of dual CD33+/CD371 events in non-T-cell (CD3−) PBMC cell types. FIG. 12B shows the percentage of each PBMC cell type population positive for both CD33 and CD371. FIG. 12B shows that a large percentage of Basophils, Myeloid cells and Monocyte cell populations identified in Example 2 demonstrate dual expression of both CD33 and CD371 and would therefore experience off-target cytotoxicity caused by a treatment targeting both CD33 and CD371. Targeting Myeloid cell populations would lead to immune suppression and specifically myelosuppression. Targeting Monocytes and Basophils would lead to reduced immunity thereby causing the patient to be more susceptible to infection and disease during the course of any treatment. Furthermore, FIG. 13A shows that there were a substantial number of dual CD33+/CD371+ detection events in BMMC cell populations from healthy patient samples. A high percentage of Myeloid progenitor cells, monocytes, Common Lymphoid Progenitor cells (CLP) and CD123+/CD38+ cells isolated from healthy patient BMMC samples demonstrate dual expression of both CD33 and CD371. Any off-target cytotoxicity directed towards these cell types leads to immune suppression and specifically myelosuppression. Therefore, any treatment that targets both CD33 and CD371 does not avoid negative off-target cytotoxicity and would lead to immune suppression and myelosuppression by simultaneously targeting BMMC cells along with any malignancy.

### Example 7: Cytotoxicity Data Demonstrating Efficient Cell Kill Using CD33-CD56 Bispecific ADCs in KASUMI-3 (CD33+CD56+) and No Cell Killing Activity in DND-39 (CD33−CD56−) Cell Lines

### Reagents

KASUMI-3 cells (DSMZ)

DND-39 cells (DSMZ)

BVX020148 (8.77 uM) (CD33+CD56+ bispecific antibody drug conjugate using mcMMAF, linker:payload (In House)

Clear bottom 96-well plates CytoOne®, Non-Treated (#CC7672-7596) (STARLABS)

Disposable PS Reservoirs-StarTub PS (#E2310-1010) (STARLABS)

CELLPRO-RO Roche Cell Proliferation Reagent WST-1 (#ab155902) (Abcam)

96-Well Clear Round Bottom 2 mL Polypropylene Deep Well Plate (#AXYPDW20CS) (SLS)

RPMI-1640 medium (#21875059) (Gibco, Life Technologies)

Foetal Bovine Serum, Heat inactivated (#11533387) (Gibco, Life Technologies)

### Methods

KASUMI-3 and DND-39 cell lines were harvested, counted and the volume required to seed 20,000 cells per well in 50 μL media calculated for a 96-well plate. A 9-point dose response of BVX020148 was prepared in Assay Media (RPMI, 10% FBS) at 2× the final concentration with a top final concentration of 104 nM. 50 μL of each dose was pipetted across duplicate wells in a 96 well plate and a separate plate was prepared for each cell line tested. 50 μL of assay media was pipetted in the blank control and in the cell only control wells and the plates incubated at 37° C., 5% CO₂ for 96 hours. After 96 hours incubation, 10 μL of WST-1 reagent was added per well and after 4 hours incubation at 37° C., 5% CO₂ the absorbance was read. The data for each reading was plotted in GraphPad PRISM and the IC50 values recorded. Data in the IC50 summary table (Table 3 below) was expressed as % cell survival in respect to the cell only control.

### Results

FIG. 14 shows the cell kill curve when DND-39 and KASUMI-3 cells were incubated with increasing concentrations of a CD33+/CD56+ antibody drug conjugate (BVX020148). DND-39 cells do not express CD33 and CD56 on their cell surface whereas KASUMI-3 cells express both CD33 and CD56 on their cell surface. As shown in FIG. 14 incubation with a CD33+/CD56+ antibody drug conjugate causes cytotoxicity at concentrations above 0.05 nM in KASUMI-3 cells. There is no observed toxicity when DND-39 cells are exposed to the same antibody drug conjugate. This result shows that the CD33+/CD56+ antibody drug conjugate specifically target cells expressing both CD33 and CD56. The IC50 values for the CD33+/CD56+ antibody drug conjugate follow this pattern and are shown in Table 3 below.

TABLE 3
Mean IC50s of CD7/CD33 bispecific ADC calculated in KASUMI-3
(CD33+CD56+) and DND-39 (CD33−CD56−) cell lines
across multiple experiments.

| | KASUMI-3 | DND-39 |
|---|---|---|
| Mean IC50 when treated with | 0.11 | >100 |
| BVX020148 (nM) | | |
| Number of Experiments | 2 | 2 |

### Example 8: Colony Forming Unit Assay Demonstrating CD33-CD56 Bispecific ADCs do not Affect the Ability of Healthy Primary Human to Differentiate from CD34+ Progenitor Cells to Myeloid CD33+ Cells

### Reagents

BVX020148 (8.77 μM) (CD33+CD56+ bispecific antibody drug conjugate using mcMMAF linker:payload (In House)

α-Fab-MMAF (13.3 μM) (Moradec #AH-121AF-50)

Gemtuzumab (6.8 μM) (In House)

Human CD34+ Cells (hCD34+-CB-c single donor) (Stemcell Technologies #70008.4)

Express MethoCult™ (Stemcell Technologies #04437)

Iscove's MDM with 2% FBS (Stemcell Technologies #7700)

SmartDish (Stemcell Technologies #27370)

STEMgrid™-6 (Stemcell Technologies #27000)

### Methods

Methocult was thawed at 4° C. overnight. Methocult was shaken and allowed to stand at room temperature until all the bubbles had dispersed and aliquoted into 3 mL aliquots. A 6-point dose titration was prepared for each antibody in PBS to achieve the following final concentrations: 10, 3, 1, 0.1, 0.01 and 0 nM. Enough of each dilution was prepared to test activity across duplicate wells. Each titration was pipetted into a 1.5 mL tube. CD34+ cells were thawed at 37° C. and suspended in 2% FBS IMDM. 300 μL of suspension containing 3000 CD34+ cells were pipetted into each 1.5 mL tube containing antibody titration and the solution gently was mixed by pipetting. For the condition Gemtuzumab+α-Fab-MMAF, Gemtuzumab was incubated with the cells for 10 min at room temperature before adding α-Fab-MMAF. The total volume of antibody dilution added to the cells was 30 μL in all conditions. The cell suspension/conjugate mix was pipetted into a 3 mL aliquot of Methocult, vortexed for 5 seconds and allowed to stand at room temperature until the bubbles had disappeared. Using an 18-gauge blunt needle and 6 mL syringe, 1 mL of cell/conjugate/Methocult mix was transferred to a well of a Smart dish and each condition was tested across duplicate wells. Each plate was rocked to ensure the Methocult covered the entire well surface evenly and then placed in a 37° C., 5% CO₂ incubator. The colonies were counted on day 9 and day 14 and the data plotted in Excel. This experiment was repeated twice using the same healthy human donor.

### Results (Day 9, Experiment 1)

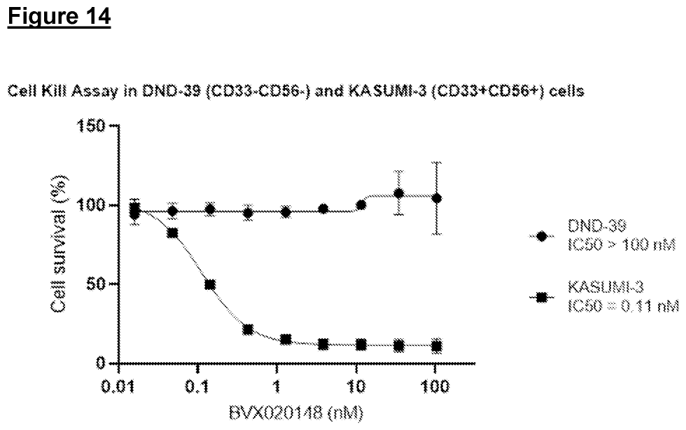

FIG. 15 shows that incubation with a bispecific CD33+/CD56+ antibody drug conjugate for 9 days does not lead to a large reduction in differentiation of healthy human CD34+ progenitor cells to myeloid CD33+ cells and CD33+ colonies. In contrast to this, conjugated Gemtuzumab (CD33 monospecific) causes a large decrease in myeloid CD33+ colony forming units during differentiation of healthy human CD34+ progenitor cells over the same time period. An overview of the colony forming unit assay data for day 9 is shown in Table 4 below. This data is summarised in terms of percentage of colonies surviving at day in Table 5 below.

TABLE 4
Colony count data from Experiment 1 used to plot
average colony count at day 9 in FIG. 15.

| | CONCEN- | | AVERAGE |
|---|---|---|---|
| REAGENT | TRATION | COLONY | COLONY |
| TESTED | (NM) | COUNT | COUNT |
| CONTROL | Cell only_1 | 53 | 53.8 |
| | Cell only_2 | 45 | |
| | Cell only_3 | 62 | |
| | Cell only_4 | 55 | |
| GEMTUZUMAB | 3_1 | 48 | 46 |
| | 3_2 | 44 | |
| | 10_1 | 60 | 55 |
| | 10_2 | 50 | |
| AFAB-MMAF | 0.01_1 | 54 | 51 |
| | 0.01_2 | 48 | |
| | 0.1_1 | 48 | 46.5 |
| | 0.1_2 | 45 | |
| | 1_1 | 63 | 58 |
| | 1_2 | 53 | |
| | 3_1 | 51 | 48 |
| | 3_2 | 45 | |
| | 10_1 | 49 | 45.5 |
| | 10_2 | 42 | |
| GEMTUZUMAB + | 0.01_1 | 55 | 52 |
| AFAB-MMAF | 0.01_2 | 49 | |
| | 0.1_1 | 39 | 39 |
| | 0.1_2 | 39 | |
| | 1_1 | 47 | 44 |
| | 1_2 | 41 | |
| | 3_1 | 42 | 39 |
| | 3_2 | 36 | |
| | 10_1 | 18 | 15.5 |
| | 10_2 | 13 | |
| BVX020148 | 0.01_1 | 45 | 47.5 |
| | 0.01_2 | 50 | |
| | 0.1_1 | 50 | 39.5 |
| | 0.1_2 | 29 | |
| | 1_1 | 37 | 36.5 |
| | 1_2 | 36 | |
| | 3_1 | 50 | 43 |
| | 3_2 | 36 | |
| | 10_1 | 34 | 36 |
| | 10_2 | 38 | |

TABLE 5
Percentage of colonies surviving after 9 days of incubation
with either Gemtuzumab, conjugated Gemtuzumab, BVX020148
or the cytotoxic payload only (Experiment 1).

| | Av. % of Colonies Surviving |
|---|---|
| Reagent | at 10 nM vs Control |
| BVX020148 | 66.98% |
| GEMTUZUMAB | 102.33% |
| αFab-MMAF | 84.65% |
| GEMTUZUMAB + | 28.84% |
| αFab-MMAF | |

The percentage of CD33+ colonies surviving after 9 days of incubation with a conjugated CD33+/CD56+ bispecific antibody is 66.98% compared to the control. Only 28.84% of CD33+ colonies survive after 9 days when incubated with conjugated Gemtuzumab. From these results it is clear that targeting CD33 and CD56 with a bispecific antibody conjugated to a cytotoxic payload is an effective way to cause cytotoxicity in cancerous cells expressing both CD33 and CD56 (KASUMI-3 cells) while reducing the off-target negative effects in differentiation of CD34+ myeloid progenitor cells into CD33+ colonies.

### Results (Day 14, Experiment 1)

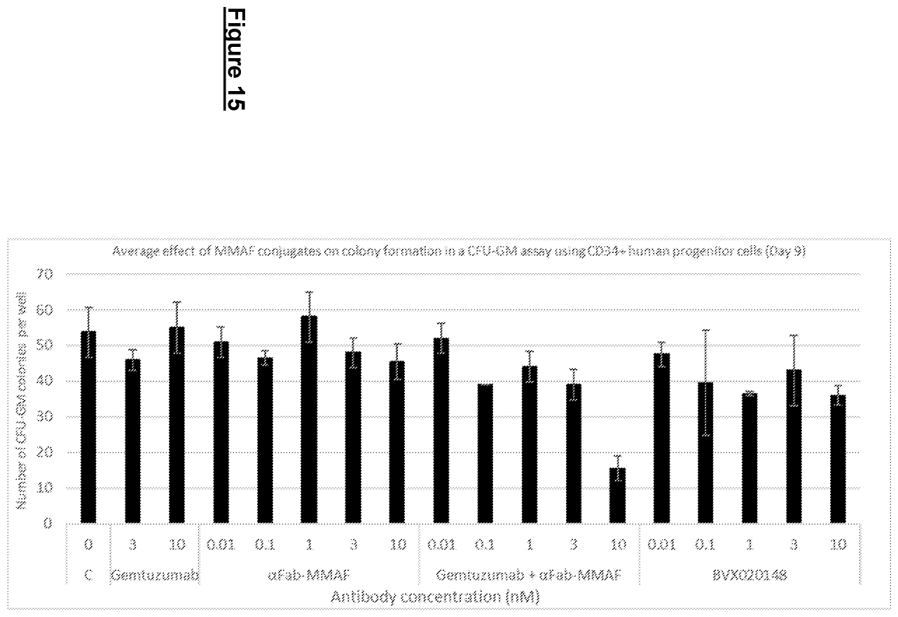

The same assay as for 9 days was carried out at 14 days (FIG. 16, Table 6 and Table 7 below). The reduction in colony formation at 14 days (FIG. 16) broadly matches the results seen after 9 days of incubation with various compositions. Although there is a slightly increased reduction in colony forming units when BVX020148 is incubated with cells at 14 days compared to 9 days the reduction in colony forming units is not as high as those cells incubated with conjugated Gemtuzumab.

TABLE 6
Colony count data from Experiment 1 used to plot
average colony count at day 14 in FIG. 16.

| | CONCEN- | | AVERAGE |
|---|---|---|---|
| REAGENT | TRATION | COLONY | COLONY |
| TESTED | (NM) | COUNT | COUNT |
| CONTROL | Cell only_1 | 44 | 52.8 |
| | Cell only_2 | 42 | |
| | Cell only_3 | 65 | |
| | Cell only_4 | 60 | |
| GEMTUZUMAB | 3_1 | 57 | 51 |
| | 3_2 | 45 | |
| | 10_1 | 53 | 53 |
| | 10_2 | 53 | |
| AFAB-MMAF | 0.01_1 | 57 | 54.5 |
| | 0.01_2 | 52 | |
| | 0.1_1 | 48 | 47 |
| | 0.1_2 | 46 | |
| | 1_1 | 58 | 51 |
| | 1_2 | 44 | |
| | 3_1 | 47 | 46 |
| | 3_2 | 45 | |
| | 10_1 | 47 | 40 |
| | 10_2 | 33 | |
| GEMTUZUMAB + | 0.01_1 | 54 | 52.5 |
| AFAB-MMAF | 0.01_2 | 51 | |
| | 0.1_1 | 45 | 43.5 |
| | 0.1_2 | 42 | |
| | 1_1 | 49 | 43.5 |
| | 1_2 | 38 | |
| | 3_1 | 48 | 41 |
| | 3_2 | 34 | |
| | 10_1 | 16 | 14.5 |
| | 10_2 | 13 | |
| BVX020148 | 0.01_1 | 66 | 61.5 |
| | 0.01_2 | 57 | |
| | 0.1_1 | 64 | 55 |
| | 0.1_2 | 46 | |
| | 1_1 | 48 | 48.5 |
| | 1_2 | 49 | |
| | 3_1 | 63 | 56.5 |
| | 3_2 | 50 | |
| | 10_1 | 36 | 34.5 |
| | 10_2 | 33 | |

TABLE 7
Percentage of colonies surviving after 14 days of incubation
with either Gemtuzumab, conjugated Gemtuzumab, BVX020148
or the cytotoxic payload only (Experiment 1).

| | Av. % of Colonies Surviving |
|---|---|
| Reagent | at 10 nM vs Control |
| BVX020148 | 65.4% |
| GEMTUZUMAB | 100.5% |
| αFab-MMAF | 75.8% |
| GEMTUZUMAB + | 27.5% |
| αFab-MMAF | |

The percentage of CD33+ colonies surviving after 14 days of incubation with a conjugated CD33+/CD56+ bispecific antibody (BVX020148) is 65.40% compared to the control. Only 27.50% of CD33+ colonies survive after 14 days when incubated with conjugated Gemtuzumab. From these results it is clear that targeting CD33 and CD56 with a bispecific antibody conjugated to a cytotoxic payload is an effective way to cause cytotoxicity in cancerous cells expressing both CD33 and CD56 (KASUMI-3 cells) while reducing the off-target negative effects in differentiation of CD34+ myeloid progenitor cells into CD33+ colonies.

### Results (Day 9, Experiment 2)

The results of the second experiment follow the patterns that were seen in the first experiment. Although there was a reduction in CD33+ colony forming units when a conjugated CD33+/CD56+ bispecific antibody (BVX020148) was incubated with CD34+ progenitor cells this reduction was not as large when compared to the reduction in colony forming units when incubated with conjugated Gemtuzumab. An overview of the colony forming unit assay data for day 9, Experiment 2 is shown in Table 8 below. This data is summarised in terms of percentage of colonies surviving at day in Table 9 below.

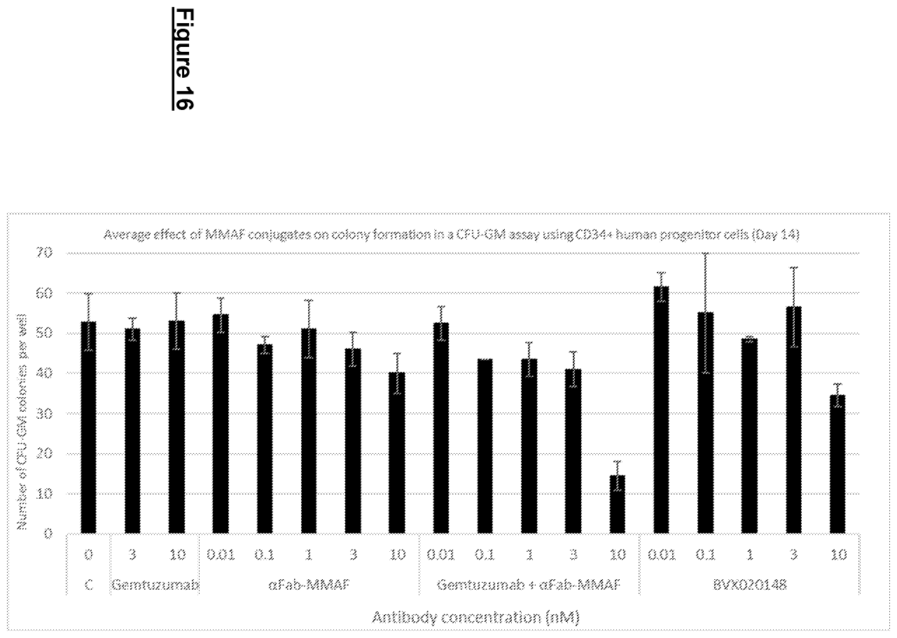

TABLE 8
Colony count data from Experiment 2 used to plot
average colony count at day 9 in FIG. 17.

| | CONCEN- | | AVERAGE |
|---|---|---|---|
| REAGENT | TRATION | COLONY | COLONY |
| TESTED | (NM) | COUNT | COUNT |
| CONTROL | Cell only_1 | 30 | 30 |
| | Cell only_2 | 30 | |
| | Cell only_3 | 28 | |
| | Cell only_4 | 32 | |
| GEMTUZUMAB | 3_1 | 33 | 33.5 |
| | 3_2 | 34 | |
| | 10_1 | 37 | 34 |
| | 10_2 | 31 | |
| AFAB-MMAF | 0.01_1 | 20 | 22.5 |
| | 0.01_2 | 25 | |
| | 0.1_1 | 25 | 24.5 |
| | 0.1_2 | 24 | |
| | 1_1 | 27 | 31.5 |
| | 1_2 | 36 | |
| | 3_1 | 30 | 26 |
| | 3_2 | 22 | |
| | 10_1 | 28 | 29.5 |
| | 10_2 | 31 | |
| GEMTUZUMAB + | 0.01_1 | 34 | 32.5 |
| AFAB-MMAF | 0.01_2 | 31 | |
| | 0.1_1 | 31 | 35.5 |
| | 0.1_2 | 40 | |
| | 1_1 | 42 | 36 |
| | 1_2 | 30 | |
| | 3_1 | 23 | 22 |
| | 3_2 | 21 | |
| | 10_1 | 8 | 10 |
| | 10_2 | 12 | |
| BVX020148 | 0.01_1 | 32 | 32 |
| | 0.01_2 | 32 | |
| | 0.1_1 | 28 | 27.5 |
| | 0.1_2 | 27 | |
| | 1_1 | 26 | 29 |
| | 1_2 | 32 | |
| | 3_1 | 27 | 26 |
| | 3_2 | 25 | |
| | 10_1 | 17 | 18 |
| | 10_2 | 19 | |

TABLE 9
Percentage of colonies surviving after 9 days of incubation
with either Gemtuzumab, conjugated Gemtuzumab, BVX020148
or the cytotoxic payload only (Experiment 2).

| | Av. % of Colonies Surviving |
|---|---|
| Reagent | at 10 nM vs Control |
| BVX020148 | 60.0% |
| GEMTUZUMAB | 113.3% |
| αFab-MMAF | 98.3% |
| GEMTUZUMAB + | 33.3% |
| αFab-MMAF | |

The percentage of CD33+ colonies surviving after 9 days of incubation with a conjugated CD33+/CD56+ bispecific antibody (BVX020148) is 60.00% compared to the control. Only 33.30% of CD33+ colonies survive after 9 days when incubated with conjugated Gemtuzumab. From these results it is clear that targeting CD33 and CD56 with a bispecific antibody conjugated to a cytotoxic payload is an effective way to cause cytotoxicity in cancerous cells expressing both CD33 and CD56 (KASUMI-3 cells) while reducing the off-target negative effects in differentiation of CD34+ myeloid progenitor cells into CD33+ colonies.

### Results (Day 14, Experiment 2)

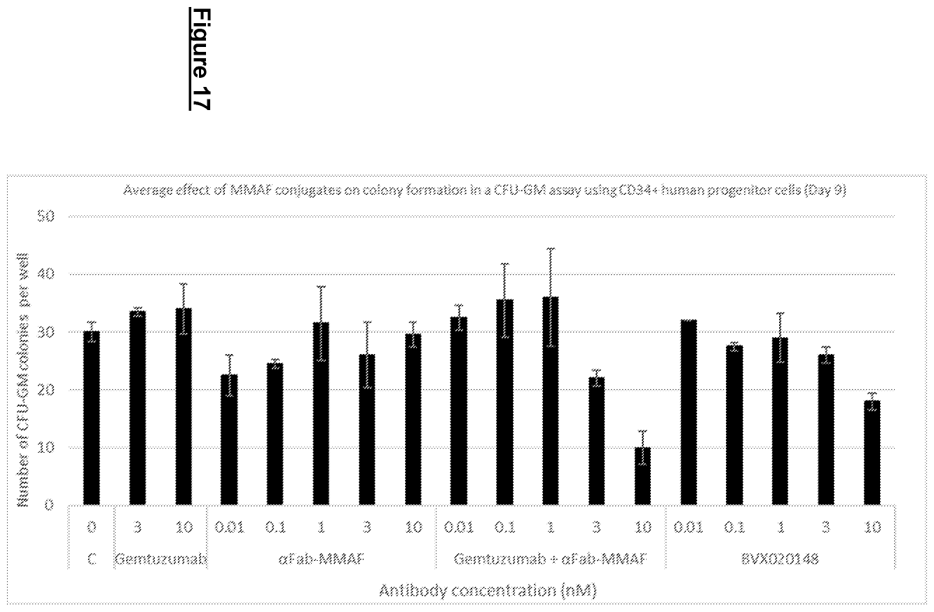

The same assay as for 9 days was carried out at 14 days (FIG. 18, Table 10 and Table 11 below). The reduction in colony formation at 14 days (FIG. 18) broadly matches the results seen after 9 days of incubation with various compositions. Although there is a reduction in colony forming units when BVX020148 is incubated with cells at 14 days this reduction is lower than the reduction seen when CD34+ cells are incubated with conjugated Gemtuzumab.

TABLE 10
Colony count data from Experiment 2 used to plot
average colony count at day 14 in FIG. 18.

| | CONCEN- | | AVERAGE |
|---|---|---|---|
| REAGENT | TRATION | COLONY | COLONY |
| TESTED | (NM) | COUNT | COUNT |
| CONTROL | Cell only_1 | 49 | 50.8 |
| | Cell only_2 | 47 | |
| | Cell only_3 | 51 | |
| | Cell only_4 | 56 | |
| GEMTUZUMAB | 3_1 | 39 | 45 |
| | 3_2 | 51 | |
| | 10_1 | 41 | 42 |
| | 10_2 | 43 | |
| AFAB-MMAF | 0.01_1 | 36 | 39.5 |
| | 0.01_2 | 43 | |
| | 0.1_1 | 40 | 39 |
| | 0.1_2 | 38 | |
| | 1_1 | 40 | 41.5 |
| | 1_2 | 43 | |
| | 3_1 | 42 | 40 |
| | 3_2 | 38 | |
| | 10_1 | 39 | 37.5 |
| | 10_2 | 36 | |
| GEMTUZUMAB + | 0.01_1 | 47 | 42.5 |
| AFAB-MMAF | 0.01_2 | 38 | |
| | 0.1_1 | 43 | 47 |
| | 0.1_2 | 51 | |
| | 1_1 | 50 | 42 |
| | 1_2 | 34 | |
| | 3_1 | 31 | 36 |
| | 3_2 | 41 | |
| | 10_1 | 14 | 17.5 |
| | 10_2 | 21 | |
| BVX020148 | 0.01_1 | 40 | 43.5 |
| | 0.01_2 | 47 | |
| | 0.1_1 | 47 | 44 |
| | 0.1_2 | 41 | |
| | 1_1 | 41 | 44 |
| | 1_2 | 47 | |
| | 3_1 | 43 | 38.5 |
| | 3_2 | 34 | |
| | 10_1 | 30 | 28.5 |
| | 10_2 | 27 | |

TABLE 11
Percentage of colonies surviving after 14 days of incubation
with either Gemtuzumab, conjugated Gemtuzumab, BVX020148
or the cytotoxic payload only (Experiment 2).

| | Av. % of Colonies Surviving |
|---|---|
| Reagent | at 10 nM vs Control |
| BVX020148 | 56.2% |
| GEMTUZUMAB | 82.8% |
| αFab-MMAF | 73.9% |
| GEMTUZUMAB + | 34.5% |
| αFab-MMAF | |

### Example 9

### Fab Expression

Sequences for Fab Expression:

Gemtuzumab VH-antiCD33:
(SEQ ID NO. 3)
EVQLVQSGAEVKKPGSSVKVSCKASGYTITDSNIHWVRQAPGQSL
EWIGYIYPYNGGTDYNQKFKNRATLTVDNPTNTAYMELSSLRSED
TAFYYCVNGNPWLAYWGQGTLVTVSS
Gemtuzumab VL-antiCD33:
(SEQ ID NO. 4)
DIQLTQSPSTLSASVGDRVTIICRASESLDNYGIRFLTWFQQKPG
KAPKLLMYAASNQGSGVPSRFSGSGSGTEFTLTISSLQPDDFATY
YCQQTKEVPWSFGQGTKLEIK
VH-antiCD56:
(SEQ ID NO. 5)
EVQLVQSGAEVKKPGSSVKVSCKASGGTFTGYYMHWVRQAPGQGL
EWMGWINPNSGGTNYAQKFQGRVTMTRDTSISTAYMELSRLRSDD
TAVYYCARDLSSGYSGYFDYWGQGTLVTVSS
VL-antiCD56:
(SEQ ID NO. 6)
DVVMTQSPLSLPVTPGEPASISCRSSQSLLHSNGYNFLDWYLQKP
GQSPQLLIYLGSNRASGVPDRFSGSGSGTDFTLKISRVEADDVGV
YYCMQSLQTPWTFGHGTKVEIK

VH and VL fragments were cloned into separate mammalian expression vectors encoding for human CH1 and CL domains downstream, respectively. Transfection grade DNA was prepared using the Plasmid Plus Midi Kit (Qiagen, Cat. No. 12945) according to manufacturer's instructions.

Fabs were expressed using the Expi293F (LifeTech, Cat. No. A14525) expression system following manufacturer's instructions, and subsequently batch purified using anti-CH1 resin. Briefly, for each Fab, 100 mL of culture was transfected, incubated at 37° C., 8% CO₂, 80% humidity, and harvested by centrifugation after 6 days. The supernatant was filtered using a 0.22 μM filter (Merck, Cat. No. 15939180) and stored at 4° C. until required. 10×PBS (ThermoFisher, Cat. No. 70013032) was added at 1/10^{th} volume of the supernatant. CaptureSelect™ CH1-XL Affinity Matrix (ThermoFisher, Cat. No. 1943462005), an anti-CH1 resin, was added in an appropriate volume. The tubes were incubated overnight at 4° C. on a rotating wheel to ensure thorough mixing of the supernatant and resin. The following day the tubes were centrifuged, and the resin transferred to a Proteus ‘1-Step Batch’ Midi Spin Column (ProteinArk, Cat. No. GEN-1SB08) followed by two wash steps using wash buffer (1×PBS with 200 mM NaCl). The protein was eluted using 600 μL 0.2M Glycine pH3.0, directly neutralised in 200 μL of 1M Tris-HCl pH8.0. The protein concentration was measured by A280 reading before dialysis into 1×PBS overnight using GeBAFlex Midi Tubes, 8 kDa Cut-Off (ProteinArk, Cat. No. MD6-22-30). The protein concentrations were re-measured, quality assessed by SDS-PAGE, and subsequently stored at 4° C. ready for Bi-fab formation.

### Fab-scFv-Fc (V Format) Expression and Purification

Sequences for Fab-scFv-Fc Expression:

Gemtuzumab VH-antiCD33:
(SEQ ID NO. 7)
EVQLVQSGAEVKKPGSSVKVSCKASGYTITDSNIHWVRQAPGQSL
EWIGYIYPYNGGTDYNQKFKNRATLTVDNPTNTAYMELSSLRSED
TAFYYCVNGNPWLAYWGQGTLVTVSS
Gemtuzumab VL-antiCD33:
(SEQ ID NO. 8)
DIQLTQSPSTLSASVGDRVTIICRASESLDNYGIRFLTWFQQKPG
KAPKLLMYAASNQGSGVPSRFSGSGSGTEFTLTISSLQPDDFATY
YCQQTKEVPWSFGQGTKLEIK
scFv-antiCD56:
(SEQ ID NO. 9)
DVVMTQSPLSLPVTPGEPASISCRSSQSLLHSNGYNFLDWYLQKP
GQSPQLLIYLGSNRASGVPDRFSGSGSGTDFTLKISRVEADDVGV
YYCMQSLQTPWTFGHGTKVEIKGGGGSGGGGSGGGGSGGGGSEVQ
LVQSGAEVKKPGSSVKVSCKASGGTFTGYYMHWVRQAPGQGLEWM
GWINPNSGGTNYAQKFQGRVTMTRDTSISTAYMELSRLRSDDTAV
YYCARDLSSGYSGYFDYWGQGTLVTVSS
Knob: CH1, CH2 & CH3
(SEQ ID NO. 10)
TKGPSVFPLAPSSKSTSGGTAALGCLVKDYFPEPVTVSWNSGALT
SGVHTFPAVLQSSGLYSLSSVVTVPSSSLGTQTYICNVNHKPSNT
KVDKKVEPKSCDKTHTCPPCPAPELLGGPSVFLFPPKPKDTLMIS
RTPEVTCVVVDVSHEDPEVKFNWYVDGVEVHNAKTKPREEQYNST
YRVVSVLTVLHQDWLNGKEYKCKVSNKALPAPIEKTISKAKGQPR
EPQVYTLPPSRDELTKNQVSLWCLVKGFYPSDIAVEWESNGQPEN
NYKTTPPVLDSDGSFFLYSKLTVDKSRWQQGNVFSCSVMHEALHN
HYTQKSLSLSPGK
Hole: G4S Linker, CH2 & CH3
(SEQ ID NO. 11)
GGGGSEPKSQDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLSCAVKGFYPSDIAVEWESNGQPENN
YKTTPPVLDSDGSFFLVSKLTVDKSRWQQGNVFSCSVMHEALHNH
YTQKSLSLSPGK

The scFv fragment for the anti-CD56 antibody was cloned into a mammalian expression vector encoding the Heavy Chain (as the Hole part of the Knob-in-Hole technology). Transfection grade DNA was prepared using the Plasmid Plus Midi Kit (Qiagen, Cat. No. 12945) according to manufacturer's instructions. This was paired with DNA for two further vectors encoding the anti-CD33 Light Chain and the Heavy Chain (as the Knob part).

The Knob-In-Hole format version of the antibody was transfected and purified as described for Fabs. The only change was the use of Fastback Protein A Sepharose Resin 100 mL (Generon, Cat. No. NB-45-00036-25) in place of anti-CH1 resin. All other aspects were as described in the Fab transfection and purification. The protein concentrations were re-measured, and quality assessed by SDS-PAGE. It was determined that further polishing was required. This was performed by Ion Exchange Chromatography.

### Bi-Fab Chemistry and Conjugation

Fabs were modified to permit Bi-fab formation by bio-orthogonal reactive partners, this was undertaken using a similar method as described in ‘The renaissance of chemically generated bispecific antibodies, Szijj P, Chudasama V, Nature Reviews Chemistry, (2021), 78-92, 5(2)’. Following formation of the Bi-Fab, the molecule was conjugated with mcMMAF targeting an average DAR 4, using a similar method as described in ‘Committee for Medicinal Products for Human Use (CHMP) Assessment report BLENREP’, EMA/CHMP/414341/2020 Corr.

V format molecules were reduced with DTT and conjugated with mcMMAF targeting an average DAR 6, using a similar method as described in ‘Committee for Medicinal Products for Human Use (CHMP) Assessment report BLENREP’, EMA/CHMP/414341/2020 Corr.

CD33×CD56 Bi-Fab ADC Demonstrates Selective and Efficient Cell Killing in Double Antigen Positive (CD33⁺CD56⁺) Vs Single Antigen Positive Cells in Cytotoxicity Assay

### Reagents

| | |
|---|---|
| KASUMI-3 cells | DSMZ |
| KE-37 cells | DSMZ |
| SET-2 cells | DSMZ |
| DND-39 cells | DSMZ |
| BVX04-a0094-AB4A-1 (CD33xCD56 Bi-Fab ADC) | In House |
| Clear bottom 96-well plates CytoOne ®, Non-Treated | STARLABS |
| (#CC7672-7596) | |
| Disposable PS Reservoirs-StarTub PS (#E2310-1010) | STARLABS |
| CELLPRO-RO Roche Cell Proliferation Reagent WST-1 | Abcam |
| (#ab155902) | |
| 96-Well Round Bottom 2 mL Polypropylene Deep Well | SLS |
| Plate (#AXYPDW20CS) | |
| RPMI-1640 medium (#21875059) | Gibco, Life |
| | Technologies |
| Fetal Bovine Serum, Heat inactivated (#11533387) | Gibco, Life |
| | Technologies |

### Method

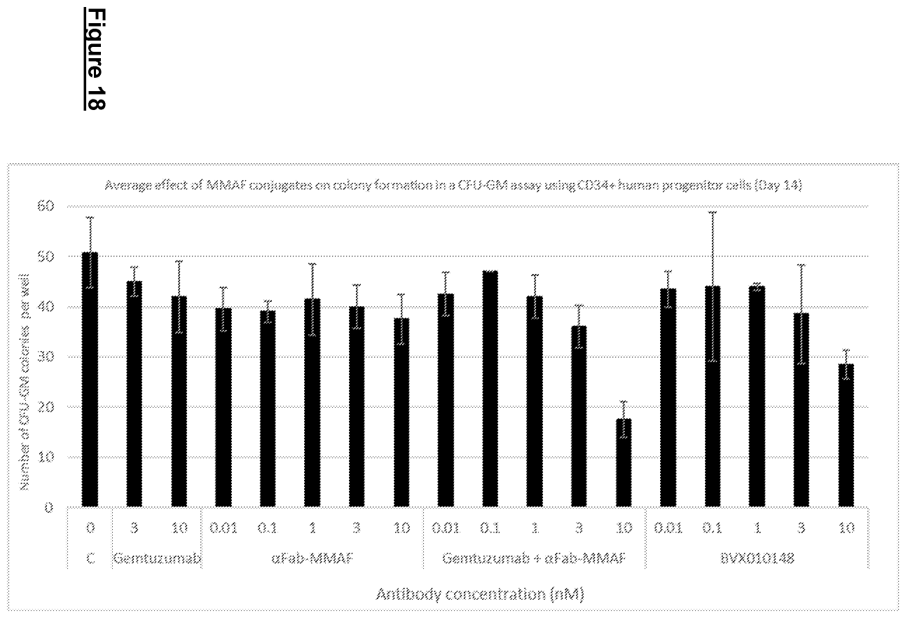

Cell lines were harvested, counted and the volume required to seed 20,000 (KASUMI-3 and KE-37), 10,000 (SET-2) and 5,000 (DND-39) cells per well in 50 μl media calculated for a 96-well plate. A 9-point dose response of ADC was prepared in assay media (RPMI, 10% FBS) at 2× the final concentration with a top final concentration of 30 nM. 50 μl of each dose was pipetted across duplicate wells in a 96 well plate and a separate plate was prepared for each cell line tested. 100 μl of assay media was pipetted in the blank control and 50 μl in the cell-only control wells; the plates incubated at 37° C., 5% CO₂ for 96 hours. After 96 hours incubation, 10 μl of WST-1 reagent was added per well and after 3 hours incubation at 37° C., 5% CO₂ the absorbance was read at 440 nm and 620 nm. The data for each reading was plotted in GraphPad PRISM and the IC50 values recorded. Data in the IC50 summary table was expressed as % cell survival in respect to the cell only control. Results are shown in FIG. 19.

TABLE 12
Mean IC50 values ± SEM of CD56/CD33 bispecific
ADC (BVX04-a0094-AB4A-1) calculated in KASUMI-3
(CD33+CD56+), KE-37 (CD56+/CD33−),
SET-2 (CD56−/CD33+) and DND-39 (CD33−CD56−) cell lines.

| | KASUMI-3 | KE-37 | SET-2 | DND-39 |
|---|---|---|---|---|
| | CD33+/ | CD33−/ | CD33+/ | CD33−/ |
| | CD56+ | CD56+ | CD56− | CD56− |
| Mean IC50 | 0.061 | >30 | >30 | >30 |
| (nM) | | | | |
| ±SEM | 0.026 | N/A | N/A | N/A |

n = 2 biological repeats (2 technical replicates per biological repeat).

### No Reduction in Healthy Human CD33⁺ Myeloid Colonies Seen in Colony Forming Unit Granulocyte-Macrophage (CFU-GM) Assay Using CD33×CD56 Bi-Fab ADC

### Reagents

| | |
|---|---|
| BVX04-a0094-AB4A-1 (CD33xCD56 | In House |
| Bi-Fab ADC) | |
| Human Cord Blood CD34+ Cells | Stemcell Technologies #70008.1 |
| (mixed donor) | |
| MethoCult ™ Optimum Without EPO | Stemcell Technologies #04437 |
| Iscove's MDM with 2% FBS | Stemcell Technologies #7700 |
| SmartDish | Stemcell Technologies #27370 |
| STEMgrid ™-6 | Stemcell Technologies #27000 |

### Method

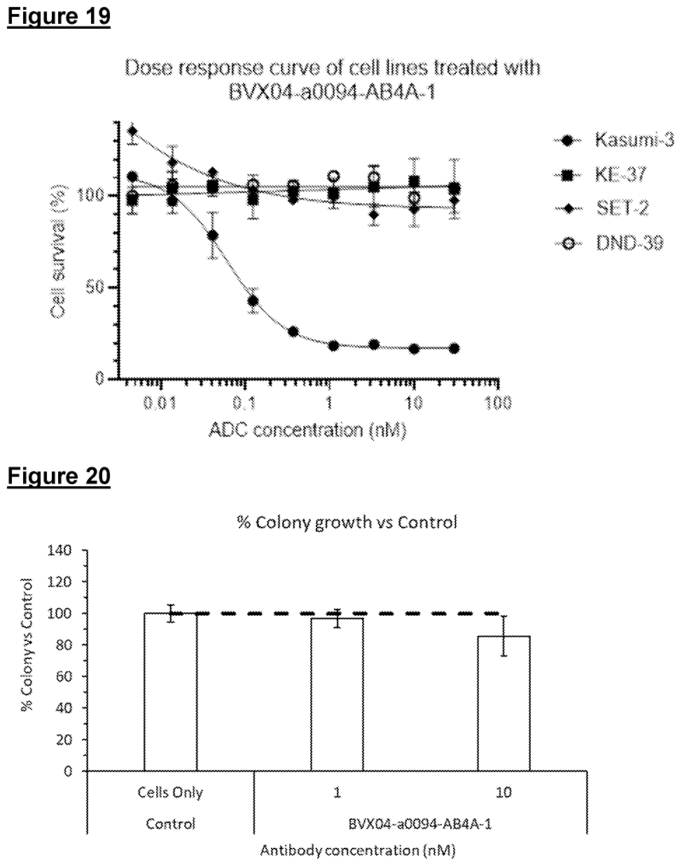

Methocult was thawed at 4° C. overnight. Methocult was shaken and allowed to stand at room temperature until bubbles had dispersed and aliquoted into 3 ml aliquots. Concentrations of 1 nM and 10 nM were prepared for each ADC in PBS. Each concentration was tested across duplicate wells. CD34+ cells were thawed at 37° C. and suspended in 2% FBS IMDM. 270 μl of suspension containing 3000 CD34+ cells were pipetted into each 1.5 ml tube containing 30 μl ADC and the solution was gently mixed by pipetting. The cell suspension/ADC mix was pipetted into a 3 ml aliquot of Methocult, vortexed for 5 seconds and allowed to stand at room temperature until the bubbles had disappeared. Using an 18-gauge blunt needle and 6 ml syringe, 1 ml of cell/ADC/Methocult mix was transferred to a well of a Smart dish. Each plate was rocked to ensure the Methocult covered the entire well surface evenly and then placed in a 37° C., 5% CO₂ incubator. The colonies were counted on day 10 and the data plotted in Excel. Results are shown in FIG. 20.

CD33×CD56 Bi-Fab ADC selectively kills dual positive (CD33⁺CD56⁺) cells compared to single antigen positive or double antigen negative cells. The CD33×CD56 Bi-Fab ADC does not cause any decrease in colony formation of CD33⁺ healthy human myeloid cells, derived from healthy human cord blood donors, within CFU-GM assay (industry standard assay for assessing risk for myelosuppression in the clinic).

CD33×CD56 V Format ADC Demonstrates Selective and Efficient Cell Killing in Double Antigen Positive (CD33⁺CD56⁺) Vs Single Antigen Positive Cells in Cytotoxicity Assay

### Reagents

| | |
|---|---|
| KASUMI-3 cells | DSMZ |
| KE-37 cells | DSMZ |
| SET-2 cells | DSMZ |
| DND-39 cells | DSMZ |
| BVX04-b0097-AB6A-1 (CD33xCD56 V format ADC) | In House |
| Clear bottom 96-well plates CytoOne ®, Non-Treated | STARLABS |
| (#CC7672-7596) | |
| Disposable PS Reservoirs-StarTub PS (#E2310-1010) | STARLABS |
| CELLPRO-RO Roche Cell Proliferation Reagent WST-1 | Abcam |
| (#ab155902) | |
| 96-Well Round Bottom 2 mL Polypropylene Deep Well | SLS |
| Plate (#AXYPDW20CS) | |
| RPMI-1640 medium (#21875059) | Gibco, Life |
| | Technologies |
| Fetal Bovine Serum, Heat inactivated (#11533387) | Gibco, Life |
| | Technologies |

### Method

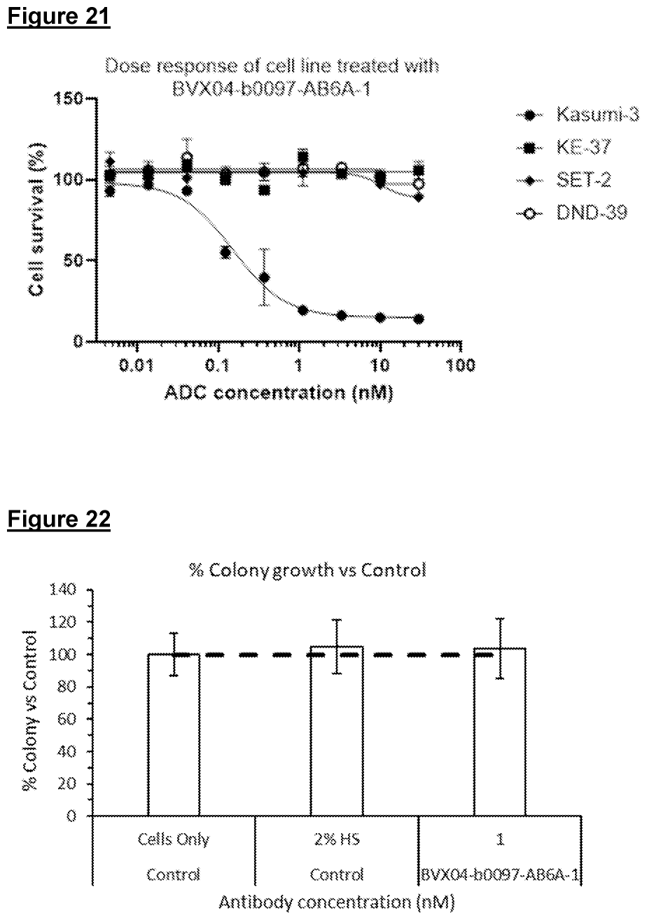

Cell lines were harvested, counted and the volume required to seed 20,000 (KASUMI-3 and KE-37), 10,000 (SET-2) and 5,000 (DND-39) cells per well in 50 μl media calculated for a 96-well plate. A 9-point dose response of ADC was prepared in assay media (RPMI, 10% FBS) at 2× the final concentration with a top final concentration of 30 nM. 2× Human serum was added to prevent non-specific Fc-internalisation. 50 μl of each dose was pipetted across duplicate wells in a 96 well plate and a separate plate was prepared for each cell line tested. 100 μl of assay media was pipetted in the blank control and 50 μl in the cell only control wells and the plates incubated at 37° C., 5% CO₂ for 96 hours. After 96 hours incubation, 10 μl of WST-1 reagent was added per well and after 3 hours incubation at 37° C., 5% CO₂ the absorbance was read at 440 nm and 620 nm. The data for each reading was plotted in GraphPad PRISM and the IC50 values recorded. Data in the IC50 summary table was expressed as % cell survival in respect to the cell only control. Results are shown in FIG. 21.

TABLE 13
Mean IC50 values ± SEM of CD56xCD33 bispecific ADC
(BVX04-b0097-AB6A-1) calculated in KASUMI-3 (CD33⁺CD56⁺),
KE-37 (CD56⁺/CD33⁻), SET-2 (CD56⁻/CD33⁺)
and DND-39 (CD33⁻CD56⁻) cell lines.

| | KASUMI-3 | KE-37 | SET-2 | DND-39 |
|---|---|---|---|---|
| | CD33⁺/ | CD33⁻/ | CD33⁺/ | CD33⁻/ |
| | CD56⁺ | CD56⁺ | CD56⁻ | CD56⁻ |
| Mean IC50 | 0.15 | >30 | >30 | >30 |
| (nM) | | | | |
| ±SEM | 0.005 | N/A | N/A | N/A |

n = 2 biological repeats (2 technical replicates per biological repeat).

### No Reduction in Healthy Human CD33⁺ Myeloid Colonies Seen in Colony Forming Unit Granulocyte-Macrophage (CFU-GM) Assay Using CD33×CD56 V Format ADC

### Reagents

| | |
|---|---|
| BVX04-b0097-AB6A-1 (CD33xCD56 | In House |
| V format ADC) | |
| Human Cord Blood CD34+ Cells | Stemcell Technologies #70008.1 |
| (mixed donor) | |
| MethoCult ™ Optimum Without EPO | Stemcell Technologies #04437 |
| Iscove's MDM with 2% FBS | Stemcell Technologies #7700 |
| SmartDish | Stemcell Technologies #27370 |
| STEMgrid ™-6 | Stemcell Technologies #27000 |

### Method

Methocult was thawed at 4° C. overnight. Methocult was shaken and allowed to stand at room temperature until bubbles had dispersed and aliquoted into 3 ml aliquots. 1 nM of ADC was prepared (BVX04-b0097-AB6A-1) in PBS and tested across duplicate wells. CD34⁺ cells were thawed at 37° C. and suspended in 2% FBS IMDM. 270 μl of suspension containing 3000 CD34⁺ cells were pipetted into each 1.5 ml tube containing 30 μl ADC and the solution was gently mixed by pipetting. The media was supplemented with 2% human serum to avoid any non-specific uptake of the ADC via Fc receptor internalisation. The cell suspension/ADC mix was pipetted into a 3 ml aliquot of Methocult, vortexed for 5 seconds and allowed to stand at room temperature until the bubbles had disappeared. Using an 18-gauge blunt needle and 6 ml syringe, 1 ml of cell/ADC/Methocult mix was transferred to a well of a Smart dish. Each plate was rocked to ensure the Methocult covered the entire well surface evenly and then placed in a 37° C., 5% CO₂ incubator. The colonies were counted on day 10 and the data plotted in Excel. Results are shown in FIG. 22.

CD33×CD56 V format ADC selectively kills dual positive (CD33+CD56+) cells compared to single antigen positive or double antigen negative cells. The CD33×CD56 V format ADC does not cause any decrease in colony formation of CD33+ healthy human myeloid cells, derived from healthy human cord blood donors, within CFU-GM assay (industry standard assay for assessing risk for myelosuppression in the clinic).

### Summary of Examples

An antigen pair was considered selective for a malignancy over healthy haematological cells and favourable based on the following criteria:
Co-expression of the antigen pair was not seen at a frequency of >30% across each individual healthy cell population screened.
Co-expression of the antigen was not found at >10% in each individual health cell population screened belonging to the following cell types: haematopoietic stem cells (HSCs), early progenitor cells, myeloid cell populations, T-cell populations and B-cell populations in more than one donor tested

Based on these criteria, the CD33×CD56 antigen pair was selected as favourable.

### SUMMARY

The expression of specific cell surface protein pairs on cancerous cells allows the effective targeting of the cancerous cells using a cytotoxic composition that binds to both proteins of such a protein pair. However, the finding that a number of these protein pairs are also expressed on certain healthy haematological cells (PBMC and BMMC cells) shows that it is not possible to easily determine which cell surface protein pairs would avoid any off-target cytotoxicity. Off-target cytotoxicity that targets healthy PBMC and/or BMMC cells leads to immune suppression and/or myelosuppression and/or impaired immune function which are common side effects of anti-cancer chemotherapy. Targeting a protein pair in which both cell surface proteins are expressed on cancerous cells but both cell surface proteins are not expressed on healthy haematological cells avoids any off-target cytotoxicity of these healthy cells. This reduces immune suppression and/or myelosuppression and/or impairment of immune function.

The experiments indicated that cell inhibiting agents targeting malignant cells expressing CD33 and CD56 could prove useful treatments as they would be non-myelosuppressing and/or non-immune suppressing. This is particularly advantageous for antibody-based therapies where myelosuppression and/or impaired immune function is often a major factor. In contrast, the experiments also indicated that cell inhibiting agents targeting malignant cells expressing CD25 and CD34; or CD56 and CD7; or CD56 and CD11c; or CD33 and CD371 would not be suitable as treatments for targeting malignant cells expressing those antigens as such targeting would result in immune suppression and/or myelosuppression and/or impaired immune function.

Using the methods detailed above it was possible to determine which cell surface protein pairs are expressed on cancerous cells but not healthy PBMC or BMMC cells isolated from healthy human patients. Having shown the cell surface protein expression pattern a conjugated bispecific antibody targeting CD33+/CD56+ showed preferential cytotoxicity for cells expressing both CD33 and CD56 over cells expressing neither of these cell surface proteins or cells expressing only one of these proteins.

A conjugated antibody targeting both CD33 and CD56 also reduced the off-target cytotoxicity observed in a CD34+ to CD33+ myeloid differentiation colony forming assay compared to a conjugated antibody that targets a single antigen. This provides further evidence that a composition e.g. bispecific antibody, targeting both CD33 and CD56 would avoid any off-target immune and/or myelosuppression in a patient receiving this composition. It is also possible to use the methods above to screen patients with a confirmed malignancy to determine whether their healthy haematological cells express both cell surface proteins that are targeted by a specific composition designed to target a protein pair expressed on the cell surface of a cancerous cell.

The forgoing embodiments are not intended to limit the scope of the protection afforded by the claims, but rather to describe examples of how the invention may be put into practice.

1. A composition for use in the treatment of a malignancy, wherein the composition comprises an agent that binds to CD33 and CD56.

2. The composition for use according to claim 1, wherein the treatment is a non-immune suppressing treatment of the malignancy, optionally selected from a non-myelosuppressing treatment.

3. The composition for use according to any preceding claim, wherein the agent is an antibody or antigen binding fragment thereof.

4. The composition for use according to claim 3, wherein the agent is a bispecific antibody or antigen binding fragment thereof that binds CD33 and CD56.

5. The composition for use according to a preceding claim, wherein the composition further comprises a payload.

6. The composition for use according to claim 5 wherein the payload is a cell killing agent, an immune-modulating payload, a macrophage class switching agent or a light activatable payload.

7. The composition for use according to claim 6 wherein the immune-modulating payload is a STING agonist or a toll-like receptor agonist.

8. The composition for use according to claim 7, wherein the cell killing agent comprises a cytotoxin.

9. The composition for use according to claim 6, wherein said cytotoxin is selected from:
i) a peptide toxin; or
ii) a chemical toxin.

10. The composition for use according to claims 5 to 9, wherein the composition further comprises a linker for linking the payload to the agent that binds to CD33 and CD56 expressed on the cell surface.

11. The composition for use according to claims 1 to 10, wherein the composition is a bispecific antibody drug conjugate.

12. The composition for use according to claims 1 to 11, wherein the malignancy is selected from one of the following cancers: haematological cancers or Multiple Myeloma.

13. The composition for use according to claims 1 to 12, wherein the malignancy is an Acute Myeloid Leukaemia (AML) or AML derived cancer.

14. A bispecific antibody or antibody fragment capable of binding CD33 and CD56 for use in the treatment of cancer.

15. The bispecific antibody according to claim 14 wherein the cancer is a haematological cancer or Multiple Myeloma.

16. The bispecific antibody according to claim 14 or 15 wherein said use comprises contacting cells that express both CD33 and CD56 with the composition.

17. A method for treating a malignancy comprising administering to a subject in deed thereof an agent that binds to CD33 and CD56.

18. The method according to claim 17, wherein the treatment is a non-immune suppressing treatment.

19. The method according to claim 18, wherein the treatment is a non-myelosuppressing treatment.

20. The method according to any of claims 17 to 19, wherein the agent is an antibody or antigen binding fragment thereof.

21. The method according to claim 20, wherein the agent is a bispecific antibody or antigen binding fragment thereof that binds CD33 and CD56.

22. The method according to any of claims 17 to 21, wherein the composition further comprises a payload.

23. The method according to claim 22 wherein the payload is a cell killing agent, an immune-modulating payload, macrophage class switching agent or a light activatable payload.

24. The method according to claim 23 wherein the immune-modulating payload is a STING agonist or a toll-like receptor agonist.

25. The method according to claim 24, wherein the cell killing agent comprises a cytotoxin.

26. The method according to claim 25, wherein said cytotoxin is selected from:
i) a peptide toxin; or
ii) a chemical toxin.

27. The method according to any of claims 23 to 26, wherein the composition further comprises a linker for linking the payload to the agent that binds to CD33 and CD56 expressed on the cell surface.

28. The method according to any of claims 17 to 27, wherein the composition is a bispecific antibody drug conjugate.

29. The method according to claims 17 to 28, wherein the malignancy is selected from one of the following cancers: haematological cancers or Multiple Myeloma.

30. The method according to any of claims 17 to 25, wherein the malignancy is an AML or AML derived cancer.

31. A method of targeting cells that express both CD33 and CD56 comprising administering to a subject an agent that binds to CD33 and CD56.

32. The method of claim 31 wherein the agent is a bispecific antibody or antigen binding fragment thereof that binds CD33 and CD56 linked to a cell killing portion.

33. A combination of agents for use in the non-immune suppressing treatment of a malignancy, wherein the agents bind to CD33 and CD56.

34. The combination for use according to claim 33, the non-immune suppressing treatment is non-myelosuppressing.

35. The combination for use according to any of claims 33 to 34, wherein the agents comprise antibodies or antigen binding fragments thereof.

36. The combination for use according to claim 35, wherein the agents further comprises a payload.

37. The composition for use according to claim 36 wherein the payload is a cell killing agent, an immune-modulating payload, macrophage class switching agent or a light activatable payload.

38. The composition for use according to claim 37 wherein the immune-modulating payload is a STING agonist or a toll-like receptor agonist.

39. The combination for use according to claim 37, wherein the cell killing agent comprises a cytotoxin.

40. The combination for use according to claim 39, wherein said cytotoxin is selected from:
i) a peptide toxin; or
ii) a chemical toxin.

41. The combination for use according to claims 36 to 40, wherein the agents further comprises a linker for linking the payload to the agent that binds to CD33 and CD56 expressed on the cell surface.

42. The combination for use according to claims 33 to 41, wherein the agent is an antibody drug conjugate.

43. The combination for use according to claims 33 to 41, wherein the malignancy is selected from one of the following cancers: haematological cancers or Multiple Myeloma.

44. The combination for use according to claims 33 to 41, wherein the malignancy is an AML or AML derived cancer.

45. A bispecific antibody or antigen binding fragment thereof capable of binding CD33 and CD56.

46. The bispecific antibody or antigen binding fragment thereof according to claim 45 linked to a payload.

47. The bispecific antibody or antigen binding fragment thereof according to claim 46 wherein the payload is a cell killing agent, an immune-modulating payload, macrophage class switching agent or a light activatable payload.

48. The bispecific antibody or antigen binding fragment thereof according to claim 47 wherein the immune-modulating payload is a STING agonist or a toll-like receptor agonist.

49. The bispecific antibody or antigen binding fragment thereof according to claim 47, wherein the cell killing agent comprises a cytotoxin.

50. The bispecific antibody or antigen binding fragment thereof according to claim 49, wherein said cytotoxin is selected from:
i) a peptide toxin; or
ii) a chemical toxin.

51. A pharmaceutical composition comprising the bispecific antibody or antigen binding fragment thereof according to any of claims 45 to 50.

52. A kit comprising the bispecific antibody or antigen binding fragment thereof antibody according to any of claims 45 to 50 or a pharmaceutical composition according to claim 51.


SEQUENCE LISTING (11 sequences)

SEQ ID NO: 1 - protein, 364 aa, Homo sapiens
MPLLLLLPLLWAGALAMDPNFWLQVQESVTVQEGLCVLVPCTFFHPIPYYDKNSPVHGYWFREGAIISGDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 2 - protein, 761 aa, Homo sapiens
MLQTKDLIWTLFFLGTAVSLQVDIVPSQGEISVGESKFFLCQVAGDAKDKDISWFSPNGEKLTPNQQRISVVWNDDSSSTLTIYNANIDDAGIYKCVVTGEDGSESEATVNVKIFQKLMFKNAPTPQEFREGEDAVIVCDVVSSLPPTIIWKHKGRDVILKKDVRFIVLSNNYLQIRGIKKTDEGTYRCEGRILARGEINFKDIQVIVNVPPTIQARQNIVNATANLGQSVTLVCDAEGFPEPTMSWTKDGEQIEQEEDDEKYIFSDDSSQLTIKKVDKNDEAEYICIAENKAGEQDATIHLKVFAKPKITYVENQTAMELEEQVTLTCEASGDPIPSITWRTSTRNISSEEKTLDGHMVVRSHARVSSLTLKSIQYTDAGEYICTASNTIGQDSQSMYLEVQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVHSPPPPASASSSTPVPLSPPDTTWPLPALATTEPAKGEPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPATLGGNSASYTFVSLLFSAVTLLLLC

SEQ ID NO: 3 - protein, 116 aa, Homo sapiens [sequence omitted]

SEQ ID NO: 4 - protein, 111 aa, Homo sapiens
DIQLTQSPSTLSASVGDRVTIICRASESLDNYGIRFLTWFQQKPGKAPKLLMYAASNQGSGVPSRFSGSGSGTEFTLTISSLQPDDFATYYCQQTKEVPWSFGQGTKLEIK

SEQ ID NO: 5 - protein, 121 aa, Homo sapiens
EVQLVQSGAEVKKPGSSVKVSCKASGGTFTGYYMHWVRQAPGQGLEWMGWINPNSGGTNYAQKFQGRVTMTRDTSISTAYMELSRLRSDDTAVYYCARDLSSGYSGYFDYWGQGTLVTVSS

SEQ ID NO: 6 - protein, 112 aa, Homo sapiens
DVVMTQSPLSLPVTPGEPASISCRSSQSLLHSNGYNFLDWYLQKPGQSPQLLIYLGSNRASGVPDRFSGSGSGTDFTLKISRVEADDVGVYYCMQSLQTPWTFGHGTKVEIK

SEQ ID NO: 7 - protein, 116 aa, Homo sapiens [sequence omitted]

SEQ ID NO: 8 - protein, 111 aa, Homo sapiens; identical to SEQ ID NO: 4

SEQ ID NO: 9 - protein, 253 aa, Homo sapiens
DVVMTQSPLSLPVTPGEPASISCRSSQSLLHSNGYNFLDWYLQKPGQSPQLLIYLGSNRASGVPDRFSGSGSGTDFTLKISRVEADDVGVYYCMQSLQTPWTFGHGTKVEIKGGGGSGGGGSGGGGSGGGGSEVQLVQSGAEVKKPGSSVKVSCKASGGTFTGYYMHWVRQAPGQGLEWMGWINPNSGGTNYAQKFQGRVTMTRDTSISTAYMELSRLRSDDTAVYYCARDLSSGYSGYFDYWGQGTLVTVSS

SEQ ID NO: 10 - protein, 328 aa, Homo sapiens
TKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 237 aa, Homo sapiens
GGGGSEPKSQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK